(19) United States
(12) Patent Application Publication
(10) Pub. No.: US 2014/0248347 A1
(43) Pub. Date: Sep. 4, 2014

(54) PHARMACEUTICAL COMPOSITIONS OF N-METHYL-2-[3-((E)-2-PYRIDIN-2-YL-VINYL)-1H-INDAZOL-6-YLSULFANYL]-BENZAMIDE

(71) Applicant: PFIZER INC., New York, NY (US)

(72) Inventors: Daniel Scott Gierer, East Lyme, CT (US); James Eric Morgado, Dartmouth, MA (US); Brendan John Murphy, Quaker Hill, CT (US); Daryl Michael Simmons, East Lyme, CT (US)

(21) Appl. No.: 14/348,415

(22) PCT Filed: Sep. 26, 2012
(86) PCT No.: PCT/IB2012/055126

§ 371 Date: Mar. 28, 2014

Related U.S. Application Data
(60) Provisional application No. 61/541,525, filed on Sep. 30, 2011.

(51) Int. Cl. A61K 31/4439; A61K 9/20; C07D 401/14; A61K 9/28; A61K 31/444

(52) U.S. Cl. CPC A61K 31/4439; A61K 9/2813; A61K 9/2866; A61K 31/444; C07D 401/14; A61K 9/2054; A61K 9/2826; A61K 9/2013
USPC 424/465; 514/338; 424/474; 424/480; 514/333; 546/256

(57) ABSTRACT

The present invention relates to pharmaceutical compositions containing axitinib, which is known as N-methyl-2-[3-((E)-2-pyridin-2-yl-vinyl)-1H-indazol-6-ylsulfanyl]-benzamide or 6-[2-(methylcarbamoyl)phenylsulfanyl]-3-E-[2-(pyridin-2-yl)ethenyl]indazole, or crystalline forms thereof, that protect axitinib from degradation, including photodegradation, as well as the therapeutic use of such compositions. The present invention also relates to novel photodegradants of axitinib.

### CROSS-REFERENCE TO RELATED APPLICATIONS

This patent application claims the benefit of priority to U.S. Provisional Patent Application Ser. No. 61/541,525, filed Sep. 30, 2011, the contents of which are hereby incorporated by reference in their entireties.

### FIELD OF THE INVENTION

The present invention relates to pharmaceutical compositions containing axitinib, which is known as 6-[2-(methylcarbamoyl)phenylsulfanyl]-3-E-[2-(pyridin-2-yl)ethenyl]indazole or N-methyl-2-[3-((E)-2-pyridin-2-yl-vinyl)-1H-indazol-6-ylsulfanyl]-benzamide, or crystalline forms thereof, that protect axitinib from degradation, including photodegradation, as well as the therapeutic use of such compositions. The present invention also relates to novel photodegradants of axitinib.

### BACKGROUND OF THE INVENTION

The compound, N-methyl-2-[3-((E)-2-pyridin-2-yl-vinyl)-1H-indazol-6-ylsulfanyl]-benzamide or 6-[2-(methylcarbamoyl)phenylsulfanyl]-3-E-[2-(pyridin-2-yl)ethenyl]indazole, of the following structure:

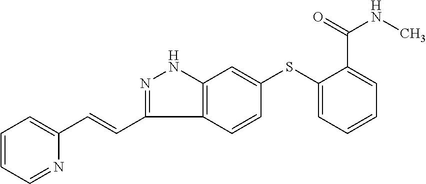 [SMILES: CNC(=O)c1ccccc1Sc1ccc2c(/C=C/c3ccccn3)n[nH]c2c1]

is known as axitinib or AG-013736.

Axitinib is a potent and selective inhibitor of vascular endothelial growth factor (VEGF) receptors 1, 2 and 3. These receptors are implicated in pathologic angiogenesis, tumor growth, and metastatic progression of cancer. Axitinib has been shown to potently inhibit VEGF-mediated endothelial cell proliferation and survival. Clinical trials are currently on-going to study the use of axitinib for the treatment of various cancers, including liver cancer, melanoma, mesothelioma, non-small cell lung cancer, prostate cancer, renal cell carcinoma, soft tissue sarcomas and solid tumors. Inlyta® (axitinib) has been approved in the United States, Europe, Japan and other jurisdictions for the treatment of renal cell carcinoma.

Axitinib, as well as pharmaceutically acceptable salts thereof, is described in U.S. Pat. No. 6,534,524. Methods of making axitinib are described in U.S. Pat. Nos. 6,884,890 and 7,232,910, in U.S. Publication Nos. 2006-0091067 and 2007-0203196 and in International Publication No. WO 2006/048745. Dosage forms of axitinib are described in U.S. Publication No. 2004-0224988. Polymorphic forms and pharmaceutical compositions of axitinib are also described in U.S. Publication Nos. 2006-0094763, 2008-0274192 and 2010-0179329. The patents and patent applications listed above are hereby incorporated by reference.

In the course of drug development, it was found that the active pharmaceutical ingredient, axitinib, was highly susceptible to degradation, including photodegradation. Successful drug development requires that patients receive the optimal dosage of an active pharmaceutical ingredient. A successful drug formulation or composition delivers the optimal dosage of an active pharmaceutical ingredient and has sufficient shelf-life to allow successful distribution to those patients in need of treatment.

While it is known to one of skill in the art that the components of tablet coatings may protect an active pharmaceutical ingredient from photodegradation, it is difficult to predict which coating excipient will provide adequate photoprotection. During formulation development for axitinib, it was found that conventional coating excipients did not protect axitinib from light. Therefore, in order to successfully develop axitinib, there was a need for a photostable pharmaceutical composition.

We have now surprisingly and unexpectedly discovered a photostable pharmaceutical composition containing axitinib.

### SUMMARY OF THE INVENTION

Each of the embodiments described below can be combined with any other embodiment described herein not inconsistent with the embodiment with which it is combined.

Some embodiments relate to a pharmaceutical composition (“Composition A”) comprising a core and a coating, the core comprising N-methyl-2-[3-((E)-2-pyridin-2-yl-vinyl)-1H-indazol-6-ylsulfanyl]-benzamide or a pharmaceutically acceptable salt thereof and excipients, and the coating comprising a metal oxide.

Further embodiments relate to the pharmaceutical composition described above, wherein the coating further comprises a filler, a polymer, a plasticizer, or an opacifier, or combinations thereof.

Additional embodiments relate to any of the embodiments of the pharmaceutical composition described above, wherein the coating further comprises a colorant.

Additional embodiments relate to any of the embodiments of the pharmaceutical composition described above, wherein the metal oxide comprises iron oxide.

Further embodiments relate to any of the embodiments of the pharmaceutical composition described above, wherein the coating is selected from the group consisting of Opadry II Red®, Opadry II Yellow®, and Opadry II Gray®.

More embodiments relate to any of the embodiments of the pharmaceutical composition described above, wherein the coating is Opadry II Red®.

Additional embodiments relate to the pharmaceutical composition described above, wherein the composition is a tablet.

Some embodiments relate to the pharmaceutical composition described above, wherein the composition is a film coated tablet.

Some embodiments relate to the pharmaceutical composition described above, wherein the composition is a capsule.

More embodiments relate to the pharmaceutical composition described above, wherein the composition is a dry-filled capsule.

Further embodiments relate to the pharmaceutical composition described above, wherein the composition is a microsphere-filled capsule.

Additional embodiments relate to the pharmaceutical composition described above, wherein the coating comprises about 4 weight percent of the composition.

Additional embodiments relate to the pharmaceutical composition described above, wherein N-methyl-2-[3-((E)-2-pyridin-2-yl-vinyl)-1H-indazol-6-ylsulfanyl]-benzamide has a mean particle of D(v, 0.5) NMT 25 microns.

Further embodiments relate to the pharmaceutical composition described above, wherein N-methyl-2-[3-((E)-2-pyridin-2-yl-vinyl)-1H-indazol-6-ylsulfanyl]-benzamide has a mean particle of D(v, 0.9) NMT 81 microns.

Some embodiments relate to a pharmaceutical composition (“Composition B”) comprising N-methyl-2-[3-((E)-2-pyridin-2-yl-vinyl)-1H-indazol-6-ylsulfanyl]-benzamide or a pharmaceutically acceptable salt thereof and excipients, wherein the pharmaceutical composition comprises at least one compound selected from the group consisting of

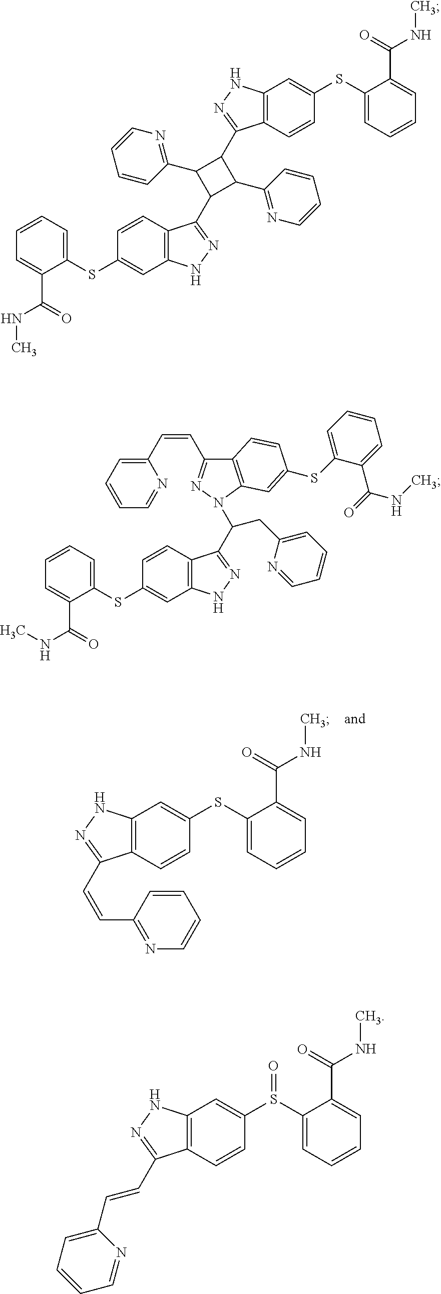 [SMILES: CNC(=O)c1ccccc1Sc1ccc2c(C3C(c4ccccn4)C(c4n[nH]c5cc(Sc6ccccc6C(=O)NC)ccc45)C3c3ccccn3)n[nH]c2c1 ; CNC(=O)c1ccccc1Sc1ccc2c(C(Cc3ccccn3)n3nc(/C=C\c4ccccn4)c4ccc(Sc5ccccc5C(=O)NC)cc43)n[nH]c2c1 ; CNC(=O)c1ccccc1Sc1ccc2c(/C=C\c3ccccn3)n[nH]c2c1 ; "and" ; CNC(=O)c1ccccc1S(=O)c1ccc2c(/C=C/c3ccccn3)n[nH]c2c1]

Additional embodiments relate to a pharmaceutical composition (“Composition C”) comprising N-methyl-2-[3-((E)-2-pyridin-2-yl-vinyl)-1H-indazol-6-ylsulfanyl]-benzamide or a pharmaceutically acceptable salt thereof and excipients, wherein the pharmaceutical composition comprises at least one compound selected from the group consisting of

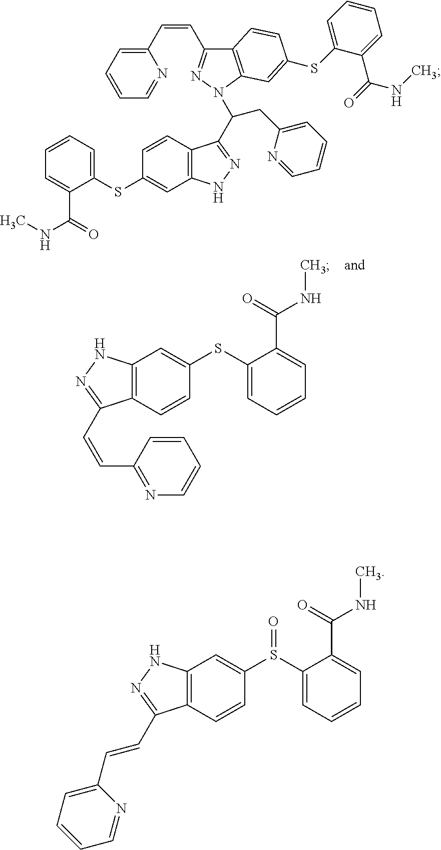 [SMILES: CNC(=O)c1ccccc1Sc1ccc2c(C(Cc3ccccn3)n3nc(/C=C\c4ccccn4)c4ccc(Sc5ccccc5C(=O)NC)cc43)n[nH]c2c1 ; CNC(=O)c1ccccc1Sc1ccc2c(/C=C\c3ccccn3)n[nH]c2c1 ; "and" ; CNC(=O)c1ccccc1S(=O)c1ccc2c(/C=C/c3ccccn3)n[nH]c2c1]

Further embodiments relate to a pharmaceutical composition (Composition D”) comprising N-methyl-2-[3-((E)-2-pyridin-2-yl-vinyl)-1H-indazol-6-ylsulfanyl]-benzamide or a pharmaceutically acceptable salt thereof and excipients, wherein the pharmaceutical composition comprises at least one compound selected from the group consisting of

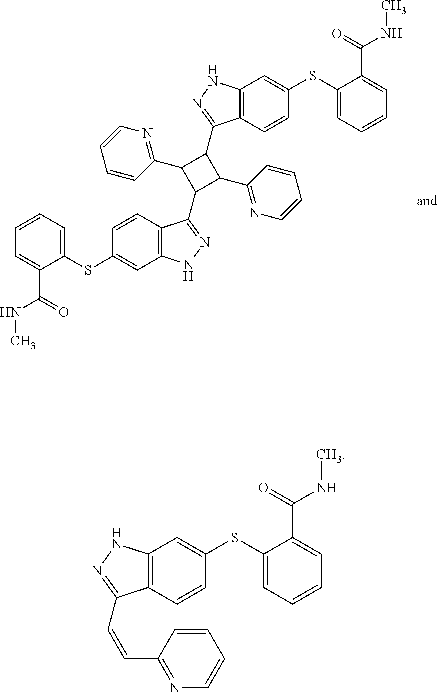 [SMILES: CNC(=O)c1ccccc1Sc1ccc2c(C3C(c4ccccn4)C(c4n[nH]c5cc(Sc6ccccc6C(=O)NC)ccc45)C3c3ccccn3)n[nH]c2c1 ; CNC(=O)c1ccccc1Sc1ccc2c(/C=C\c3ccccn3)n[nH]c2c1]

More embodiments relate to a pharmaceutical composition (“Composition E”) comprising N-methyl-2-[3-((E)-2-pyridin-2-yl-vinyl)-1H-indazol-6-ylsulfanyl]-benzamide or a pharmaceutically acceptable salt thereof and excipients, wherein the pharmaceutical composition comprises at least one compound selected from the group consisting of

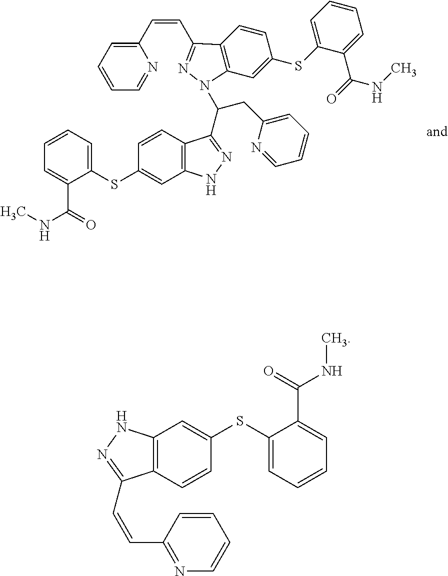

Some embodiments relate to any of Composition B, Composition C, Composition D or Composition E, wherein the pharmaceutical composition comprises less than about weight percent of the at least one compound.

More embodiments relate to any of Composition B, Composition C, Composition D or Composition E, wherein the pharmaceutical composition comprises less than about 2 weight percent of the at least one compound.

More embodiments relate to any of Composition B, Composition C, Composition D or Composition E, wherein the pharmaceutical composition comprises less than about 1 weight percent of the at least one compound.

Further embodiments relate to any of Composition B, Composition C, Composition D or Composition E, wherein the pharmaceutical composition comprises from about 0.01% weight percent to about 5 weight percent of the at least one compound.

Additional embodiments relate to any of Composition B, Composition C, Composition D or Composition E, wherein the pharmaceutical composition comprises from about 0.05% weight percent to about 5 weight percent of the at least one compound.

Additional embodiments relate to any of Composition B, Composition C, Composition D or Composition E, wherein the pharmaceutical composition comprises from about 0.01% weight percent to about 2 weight percent of the at least one compound.

More embodiments relate to any of Composition B, Composition C, Composition D or Composition E, wherein the pharmaceutical composition comprises from about 0.05% weight percent to about 2 weight percent of the at least one compound.

Some embodiments relate to a pharmaceutical composition comprising N-methyl-2-[3-((E)-2-pyridin-2-yl-vinyl)-1H-indazol-6-ylsulfanyl]-benzamide or a pharmaceutically acceptable salt thereof and excipients, wherein the pharmaceutical composition comprises less than about 1.0 weight percent of a compound, which is

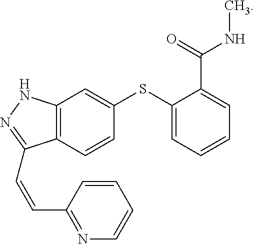 [SMILES: CNC(=O)c1ccccc1Sc1ccc2c(/C=C\c3ccccn3)n[nH]c2c1]

Further embodiments relate to a compound, which is

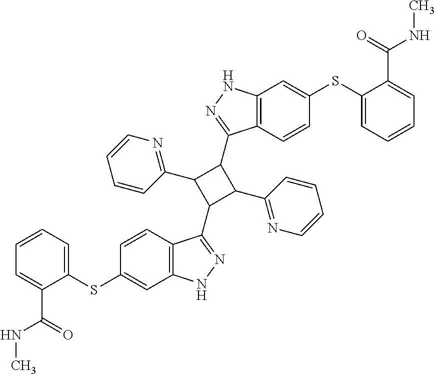 [SMILES: CNC(=O)c1ccccc1Sc1ccc2c(C3C(c4ccccn4)C(c4n[nH]c5cc(Sc6ccccc6C(=O)NC)ccc45)C3c3ccccn3)n[nH]c2c1]

or a pharmaceutically acceptable salt thereof.

Additional embodiments relate to a compound, which is

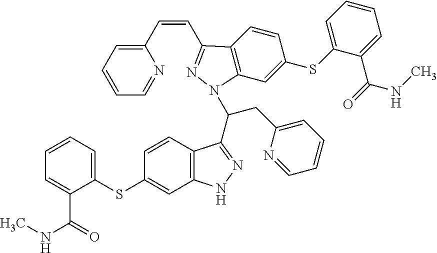

or a pharmaceutically acceptable salt thereof.

More embodiments relate to Composition A, wherein the pharmaceutical composition comprises about 1 mg of N-methyl-2-[3-((E)-2-pyridin-2-yl-vinyl)-1H-indazol-6-ylsulfanyl]-benzamide and:

a. about 89 weight percent to about 97 weight percent of at least one filler;

b. about 2 weight percent to about 5 weight percent of a disintegrant;

c. about 0.25 weight percent to about 5 weight percent of a lubricant; and

d. about 1 weight percent to about 8 weight percent of the coating, based on the total weight of the pharmaceutical composition.

Some embodiments relate to Composition A, wherein the pharmaceutical composition comprises about 1 mg of N-methyl-2-[3-((E)-2-pyridin-2-yl-vinyl)-1H-indazol-6-ylsulfanyl]-benzamide and:

a. about 92 weight percent to about 97 weight percent of at least one filler;

b. about 2 weight percent to about 4 weight percent of a disintegrant;

c. about 0.25 weight percent to about 3 weight percent of a lubricant; and

d. about 2 weight percent to about 5 weight percent of the coating, based on the total weight of the pharmaceutical composition.

Further embodiments relate to Composition A, wherein the pharmaceutical composition comprises about 3 mg of N-methyl-2-[3-((E)-2-pyridin-2-yl-vinyl)-1H-indazol-6-ylsulfanyl]-benzamide and:

a. about 87 weight percent to about 95 weight percent of at least one filler;

b. about 2 weight percent to about 5 weight percent of a disintegrant;

c. about 0.25 weight percent to about 5 weight percent of a lubricant; and

d. about 1 weight percent to about 9 weight percent of the coating, based on the total weight of the pharmaceutical composition.

Additional embodiments relate to Composition A, wherein the pharmaceutical composition comprises about 3 mg of N-methyl-2-[3-((E)-2-pyridin-2-yl-vinyl)-1H-indazol-6-ylsulfanyl]-benzamide and:

a. about 90 weight percent to about 95 weight percent of at least one filler;

b. about 2 weight percent to about 4 weight percent of a disintegrant;

c. about 0.25 weight percent to about 3 weight percent of a lubricant; and

d. about 2 weight percent to about 5 weight percent of the coating, based on the total weight of the pharmaceutical composition.

Further embodiments relate to Composition A, wherein the pharmaceutical composition comprises about 5 mg of N-methyl-2-[3-((E)-2-pyridin-2-yl-vinyl)-1H-indazol-6-ylsulfanyl]-benzamide and:

a. about 87 weight percent to about 95 weight percent of at least one filler;

b. about 2 weight percent to about 5 weight percent of a disintegrant;

c. about 0.25 weight percent to about 5 weight percent of a lubricant; and

d. about 1 weight percent to about 9 weight percent of the coating, based on the total weight of the pharmaceutical composition.

Additional embodiments relate to Composition A, wherein the pharmaceutical composition comprises about 5 mg of N-methyl-2-[3-((E)-2-pyridin-2-yl-vinyl)-1H-indazol-6-ylsulfanyl]-benzamide and:

a. about 90 weight percent to about 95 weight percent of at least one filler;

b. about 2 weight percent to about 4 weight percent of a disintegrant;

c. about 0.25 weight percent to about 3 weight percent of a lubricant; and

d. about 2 weight percent to about 5 weight percent of the coating, based on the total weight of the pharmaceutical composition.

Further embodiments relate to Composition A, wherein the pharmaceutical composition comprises about 7 mg of N-methyl-2-[3-((E)-2-pyridin-2-yl-vinyl)-1H-indazol-6-ylsulfanyl]-benzamide and:

a. about 87 weight percent to about 95 weight percent of at least one filler;

b. about 2 weight percent to about 5 weight percent of a disintegrant;

c. about 0.25 weight percent to about 5 weight percent of a lubricant; and

d. about 1 weight percent to about 9 weight percent of the coating, based on the total weight of the pharmaceutical composition.

Additional embodiments relate to Composition A, wherein the pharmaceutical composition comprises about 7 mg of N-methyl-2-[3-((E)-2-pyridin-2-yl-vinyl)-1H-indazol-6-ylsulfanyl]-benzamide and:

a. about 90 weight percent to about 95 weight percent of at least one filler;

b. about 2 weight percent to about 4 weight percent of a disintegrant;

c. about 0.25 weight percent to about 3 weight percent of a lubricant; and

d. about 2 weight percent to about 5 weight percent of the coating, based on the total weight of the pharmaceutical composition.

Further embodiments relate to Composition A, wherein the pharmaceutical composition comprises about 1 mg of N-methyl-2-[3-((E)-2-pyridin-2-yl-vinyl)-1H-indazol-6-ylsulfanyl]-benzamide and:

a. about 20 weight percent to about 90 weight percent microcrystalline cellulose;

b. about 10 weight percent to about 85 weight percent lactose monohydrate;

c. about 2 weight percent to about 5 weight percent croscarmellose sodium;

d. about 0.25 weight percent to about 5 weight percent magnesium stearate; and

e. about 1 weight percent to about 8 weight percent of the coating, based on the total weight of the pharmaceutical composition.

Further embodiments relate to Composition A, wherein the pharmaceutical composition comprises about 1 mg of N-methyl-2-[3-((E)-2-pyridin-2-yl-vinyl)-1H-indazol-6-ylsulfanyl]-benzamide and:

a. about 55 weight percent to about 70 weight percent microcrystalline cellulose;

b. about 28 weight percent to about 36 weight percent lactose monohydrate;

c. about 2 weight percent to about 4 weight percent croscarmellose sodium;

d. about 0.25 weight percent to about 3 weight percent magnesium stearate; and

e. about 2 weight percent to about 5 weight percent of the coating, based on the total weight of the pharmaceutical composition.

Additional embodiments relate to Composition A, wherein the pharmaceutical composition comprises about 3 mg of N-methyl-2-[3-((E)-2-pyridin-2-yl-vinyl)-1H-indazol-6-ylsulfanyl]-benzamide and:

a. about 20 weight percent to about 90 weight percent microcrystalline cellulose;

b. about 10 weight percent to about 85 weight percent lactose monohydrate;

c. about 2 weight percent to about 5 weight percent croscarmellose sodium;

d. about 0.25 weight percent to about 5 weight percent magnesium stearate; and

e. about 1 weight percent to about 8 weight percent of the coating, based on the total weight of the pharmaceutical composition.

Further embodiments relate to Composition A, wherein the pharmaceutical composition comprises about 3 mg of N-methyl-2-[3-((E)-2-pyridin-2-yl-vinyl)-1H-indazol-6-ylsulfanyl]-benzamide and:

a. about 55 weight percent to about 70 weight percent microcrystalline cellulose;

b. about 28 weight percent to about 36 weight percent lactose monohydrate;

c. about 2 weight percent to about 4 weight percent croscarmellose sodium;

d. about 0.25 weight percent to about 3 weight percent magnesium stearate; and

e. about 2 weight percent to about 5 weight percent of the coating, based on the total weight of the pharmaceutical composition.

Additional embodiments relate to Composition A, wherein the pharmaceutical composition comprises about 5 mg of N-methyl-2-[3-((E)-2-pyridin-2-yl-vinyl)-1H-indazol-6-ylsulfanyl]-benzamide and:

a. about 20 weight percent to about 90 weight percent microcrystalline cellulose;

b. about 10 weight percent to about 85 weight percent lactose monohydrate;

c. about 2 weight percent to about 5 weight percent croscarmellose sodium;

d. about 0.25 weight percent to about 5 weight percent magnesium stearate; and

e. about 1 weight percent to about 8 weight percent of the coating, based on the total weight of the pharmaceutical composition.

Further embodiments relate to Composition A, wherein the pharmaceutical composition comprises about 5 mg of N-methyl-2-[3-((E)-2-pyridin-2-yl-vinyl)-1H-indazol-6-ylsulfanyl]-benzamide and:

a. about 55 weight percent to about 70 weight percent microcrystalline cellulose;

b. about 28 weight percent to about 36 weight percent lactose monohydrate;

c. about 2 weight percent to about 4 weight percent croscarmellose sodium;

d. about 0.25 weight percent to about 3 weight percent magnesium stearate; and

e. about 2 weight percent to about 5 weight percent of the coating, based on the total weight of the pharmaceutical composition.

Additional embodiments relate to Composition A, wherein the pharmaceutical composition comprises about 7 mg of N-methyl-2-[3-((E)-2-pyridin-2-yl-vinyl)-1H-indazol-6-ylsulfanyl]-benzamide and:

a. about 20 weight percent to about 90 weight percent microcrystalline cellulose;

b. about 10 weight percent to about 85 weight percent lactose monohydrate;

c. about 2 weight percent to about 5 weight percent croscarmellose sodium;

d. about 0.25 weight percent to about 5 weight percent magnesium stearate; and

e. about 1 weight percent to about 8 weight percent of the coating, based on the total weight of the pharmaceutical composition.

Further embodiments relate to Composition A, wherein the pharmaceutical composition comprises about 7 mg of N-methyl-2-[3-((E)-2-pyridin-2-yl-vinyl)-1H-indazol-6-ylsulfanyl]-benzamide and:

a. about 55 weight percent to about 70 weight percent microcrystalline cellulose;

b. about 28 weight percent to about 36 weight percent lactose monohydrate;

c. about 2 weight percent to about 4 weight percent croscarmellose sodium;

d. about 0.25 weight percent to about 3 weight percent magnesium stearate; and

e. about 2 weight percent to about 5 weight percent of the coating, based on the total weight of the pharmaceutical composition.

Some embodiments further relate to any of the preceding embodiments related to Composition A, wherein the coating comprises from about 5 weight percent to about 20 weight percent of iron oxide, based on the total weight of the coating.

Additional embodiments further relate to any of the preceding embodiments related to Composition A, wherein the coating comprises about 7 weight percent of iron oxide, based on the total weight of the coating.

Further embodiments further relate to any of the preceding embodiments related to Composition A, wherein the coating comprises about 9 weight percent of iron oxide, based on the total weight of the coating.

More embodiments further relate to any of the preceding embodiments related to Composition A, wherein the coating comprises about 18 weight percent of iron oxide, based on the total weight of the coating.

Some embodiments relate to Composition A, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 8.8±0.1, 12.0±0.1, 14.5±0.1, 15.7±0.1 and 19.1±0.1.

More embodiments relate to Composition A, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a solid state nuclear magnetic resonance comprising the following ¹³C chemical shifts expressed in parts per million: 154.2±0.2, 143.3±0.2, 121.3±0.2 and 27.8±0.2.

More embodiments relate to Composition A, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a Raman spectrum comprising any one of the following Raman shifts expressed as wavenumbers in inverse centimeters: 791±2, 806±2, 850±2, 1194±2, 1242±2, 1280±2, 1309±2 and 3054±2.

Additional embodiments relate to Composition A, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 8.8±0.1 and 15.7±0.1 and a solid state nuclear magnetic resonance comprising the following ¹³C chemical shifts expressed in parts per million: 154.2±0.2, 143.3±0.2, 121.3±0.2 and 27.8±0.2.

Further embodiments relate to Composition A, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 8.8±0.1 and 15.7±0.1 and a Raman spectrum comprising any one of the following Raman shifts expressed as wavenumbers in inverse centimeters: 791±2, 806 2, 850±2, 1194±2, 1242±2, 1280±2, 1309±2 and 3054±2.

Additional embodiments relate to Composition A, wherein the N-methyl-2-[3-((E)-2-pyridin-2-yl-vinyl)-1H-indazol-6-ylsulfanyl]-benzamide is Form XXV N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 5.1±0.1, 8.0±0.1, 10.1±0.1 and 10.7±0.1.

Some embodiments relate to Composition A, wherein the N-methyl-2-[3-((E)-2-pyridin-2-yl-vinyl)-1H-indazol-6-ylsulfanyl]-benzamide is Form XXV N-methyl-2-[3-((E)-2-pyridin-2-yl-vinyl)-1H-indazol-6-ylsulfanyl]-benzamide having a solid state nuclear magnetic resonance comprising the following ¹³C chemical shifts expressed in parts per million: 128.8±0.2, 123.7±0.2, 120.5±0.2 and 25.4±0.2.

More embodiments relate to Composition A, wherein the N-methyl-2-[3-((E)-2-pyridin-2-yl-vinyl)-1H-indazol-6-ylsulfanyl]-benzamide is Form XXV N-methyl-2-[3-((E)-2-pyridin-2-yl-vinyl)-1H-indazol-6-ylsulfanyl]-benzamide having a Raman spectrum comprising any one of the following Raman shifts expressed as wavenumbers in inverse centimeters: 766±2, 822±2, 866±2, 962±2, 989±2, 1212±2, 1238±2, 1350±2, 1637±2 and 3067±2.

Further embodiments relate to Composition A, wherein the N-methyl-2-[3-((E)-2-pyridin-2-yl-vinyl)-1H-indazol-6-ylsulfanyl]-benzamide is Form XXV N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 5.1±0.1 and 10.7±0.1 and a solid state nuclear magnetic resonance comprising the following ¹³C chemical shifts expressed in parts per million: 128.8±0.2, 123.7±0.2, 120.5±0.2 and 25.4±0.2.

Additional embodiments relate to Composition A, wherein the N-methyl-2-[3-((E)-2-pyridin-2-yl-vinyl)-1H-indazol-6-ylsulfanyl]-benzamide is Form XXV N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 5.1±0.1 and 10.7±0.1 and a Raman spectrum comprising any one of the following Raman shifts expressed as wavenumbers in inverse centimeters: 766±2, 822±2, 866±2, 962±2, 989±2, 1212±2, 1238±2, 1350±2, 1637±2 and 3067±2.

Some embodiments relate to Composition A, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ10=1.54056 Å): 11.5±0.1, 11.9±0.1, 14.8±0.1 and 15.6±0.1.

More embodiments relate to Composition A, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a solid state nuclear magnetic resonance comprising the following ¹³C chemical shifts expressed in parts per million: 142.6±0.2, 133.7±0.2, 121.4±0.2 and 119.8±0.2.

More embodiments relate to Composition A, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a Raman spectrum comprising any one of the following Raman shifts expressed as wavenumbers in inverse centimeters: 835±2, 1234±2, 1564±2 and 3058±2.

Some embodiments relate to Composition A, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 11.5±0.1 and 11.9±0.1 and a solid state nuclear magnetic resonance comprising the following ¹³C chemical shifts expressed in parts per million: 142.6±0.2, 133.7±0.2, 121.4±0.2 and 119.8±0.2.

Further embodiments relate to Composition A, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 11.5±0.1 and 11.9±0.1 and a Raman spectrum comprising any one of the following Raman shifts expressed as wavenumbers in inverse centimeters: 835±2, 1234±2, 1564±2 and 3058±2.

Some embodiments relate to any of the embodiments of Composition A, wherein photodegradation of the N-methyl-2-[3-((E)-2-pyridin-2-yl-vinyl)-1H-indazol-6-ylsulfanyl]-benzamide or a pharmaceutically acceptable salt thereof, is less than about 1% as measured by the International Conference on Harmonisation of Technical Requirements for Registration of Pharmaceuticals for Human Use guideline, Q1B Photostability Testing of New Drug Substances and Products, published on November 1996.

Some embodiments relate to any of the embodiments of Composition A, wherein photodegradation of the N-methyl-2-[3-((E)-2-pyridin-2-yl-vinyl)-1H-indazol-6-ylsulfanyl]-benzamide or a pharmaceutically acceptable salt thereof, is less than about 0.05% as measured by the International Conference on Harmonisation of Technical Requirements for Registration of Pharmaceuticals for Human Use guideline, Q1B Photostability Testing of New Drug Substances and Products, published on November 1996.

Some embodiments relate to any of the embodiments of Composition A, wherein photodegradation of the N-methyl-2-[3-((E)-2-pyridin-2-yl-vinyl)-1H-indazol-6-ylsulfanyl]-benzamide or a pharmaceutically acceptable salt thereof, is less than about 0.01% as measured by the International Conference on Harmonisation of Technical Requirements for Registration of Pharmaceuticals for Human Use guideline, Q1B Photostability Testing of New Drug Substances and Products, published on November 1996.

Some embodiments relate to a method of treating abnormal cell growth in a subject comprising administering to the subject an amount of any of the embodiments of Composition A, that is effective in treating abnormal cell growth.

More embodiments relate to the method of treating abnormal cell growth, wherein the abnormal cell growth is cancer.

Additional embodiments relate to the method of treating cancer, wherein the cancer is selected from the group consisting of liver cancer, melanoma, mesothelioma, non-small cell lung cancer, prostate cancer, renal cell carcinoma, soft tissue sarcomas and solid tumors.

### BRIEF DESCRIPTION OF THE DRAWINGS

FIG. 1 shows an annotated powder X-ray diffraction pattern of axitinib Form IV in drug product carried out on a Siemens D5000 diffractometer, (λ=1.54056 Å).

FIG. 2 shows an annotated powder X-ray diffraction pattern of axitinib Form XXV in drug product carried out on a Siemens D5000 diffractometer, (λ=1.54056 Å).

FIG. 3 shows an annotated powder X-ray diffraction pattern of axitinib Form XLI in drug product carried out on a Siemens D5000 diffractometer, (λ=1.54056 Å).

FIG. 4 shows a carbon cross-polarization magic angle spinning (CPMAS) solid-state nuclear magnetic resonance spectrum of axitinib Form IV carried out on a 7 mm Bruker-Biospin CPMAS probe positioned into a wide-bore Bruker-Biospin DSX 500 MHz (¹H frequency) NMR spectrometer. The peaks marked by asterisks are spinning sidebands.

FIG. 5 shows a carbon cross-polarization magic angle spinning (CPMAS) solid-state nuclear magnetic resonance spectrum of axitinib Form IV in drug product carried out on a 7 mm Bruker-Biospin CPMAS probe positioned into a wide-bore Bruker-Biospin DSX 500 MHz (¹H frequency) NMR spectrometer. The peaks marked by asterisks are spinning sidebands.

FIG. 6 shows an annotated carbon cross-polarization magic angle spinning (CPMAS) solid-state nuclear magnetic resonance spectrum of axitinib Form IV in drug product carried out on a 7 mm Bruker-Biospin CPMAS probe positioned into a wide-bore Bruker-Biospin DSX 500 MHz (¹H frequency) NMR spectrometer. The peaks marked by asterisks are spinning sidebands.

FIG. 7 shows a carbon cross-polarization magic angle spinning (CPMAS) solid-state nuclear magnetic resonance spectrum of axitinib Form XXV in drug product carried out on a 7 mm Bruker-Biospin CPMAS probe positioned into a wide-bore Bruker-Biospin DSX 500 MHz (¹H frequency) NMR spectrometer. The peaks marked by asterisks are spinning sidebands.

FIG. 8 shows an annotated carbon cross-polarization magic angle spinning (CPMAS) solid-state nuclear magnetic resonance spectrum of axitinib Form XXV in drug product carried out on a 7 mm Bruker-Biospin CPMAS probe positioned into a wide-bore Bruker-Biospin DSX 500 MHz (¹H frequency) NMR spectrometer. The peaks marked by asterisks are spinning sidebands.

FIG. 9 shows a carbon cross-polarization magic angle spinning (CPMAS) solid-state nuclear magnetic resonance spectrum of axitinib Form XLI in drug product carried out on a 7 mm Bruker-Biospin CPMAS probe positioned into a wide-bore Bruker-Biospin DSX 500 MHz (¹H frequency) NMR spectrometer. The peaks marked by asterisks are spinning sidebands.

FIG. 10 shows an annotated carbon cross-polarization magic angle spinning (CPMAS) solid-state nuclear magnetic resonance spectrum of axitinib Form XLI in drug product carried out on a 7 mm Bruker-Biospin CPMAS probe positioned into a wide-bore Bruker-Biospin DSX 500 MHz (¹H frequency) NMR spectrometer. The peaks marked by asterisks are spinning sidebands.

FIG. 11 shows a fourier transform (FT)-Raman spectrum of axitinib Form IV carried out on a Nicolet NXR FT-Raman accessory attached to a Nicolet 6700 FTIR spectrometer.

FIG. 12 shows an annotated fourier transform (FT)-Raman spectrum of axitinib Form IV in drug product carried out on a Nicolet NXR FT-Raman accessory attached to a Nicolet 6700 FTIR spectrometer.

FIG. 13 shows an annotated fourier transform (FT)-Raman spectrum of axitinib Form XXV in drug product carried out on a Nicolet NXR FT-Raman accessory attached to a Nicolet 6700 FTIR spectrometer.

FIG. 14 shows an annotated fourier transform (FT)-Raman spectrum of axitinib Form XLI in drug product carried out on a Nicolet NXR FT-Raman accessory attached to a Nicolet 6700 FTIR spectrometer.

### DETAILED DESCRIPTION OF THE INVENTION

As used herein, “cc” means cubic centimeter, “cP” means viscosity in centipoise, “FCT” means a film coated tablet, “FT” means fourier transform, the term “grade” refers to quality or purity standards, “HPLC” means high-performance liquid chromatography, “HDPE” means high density polyethylene, “HPMC” means hydroxypropyl methylcellulose, “ICH” means the International Conference on Harmonisation of Technical Requirements for Registration of Pharmaceuticals for Human Use, “mgW” means milligram weight, “N/A” means not applicable, “No.” means number, “open” means an open shallow glass dish, “PSI” means pounds per square inch, “PXRD” means powder X-ray diffraction, “PTFE” means polytetrafluoroethylene, “QT” means quart, “tab” means tablet, “SFC” means supercritical fluid chromatography, “SSNMR” means solid-state nuclear magnetic resonance, “TLC” means thin layer chromatography, “UV” means ultraviolet, “w/w” means weight/weight, and “w/w %” means weight/weight percent.

As used herein, an “active pharmaceutical ingredient” or “API” is the biologically active substance in a pharmaceutical composition, formulation, drug product or unit dosage form. Specifically, axitinib is the active pharmaceutical ingredient in the pharmaceutical composition or drug product of the present invention.

As used herein, a “drug product” refers to a formulated active pharmaceutical ingredient. For example, a drug product may refer to a tablet or capsule that contains an active pharmaceutical ingredient and excipients. Specifically, a drug product is a pharmaceutical composition of the present invention. The terms “drug product” and “pharmaceutical composition” may be used interchangeably.

As used herein, an “effective” amount refers to an amount of a compound, agent, substance, formulation or composition that is of sufficient quantity to result in a decrease in severity of disease symptoms, an increase in frequency and duration of disease symptom-free periods, or a prevention of impairment or disability due to the disease affliction. The amount may be as a single dose or according to a multiple dose regimen, alone or in combination with other compounds, agents or substances. One of ordinary skill in the art would be able to determine such amounts based on such factors as a subject's size, the severity of a subject's symptoms, and the particular composition or route of administration selected.

The phrase “pharmaceutically acceptable salt(s)”, as used herein, unless otherwise indicated, includes salts of basic groups which may be present in axitinib. Axitinib is basic in nature and capable of forming a wide variety of salts with various inorganic and organic acids. The acids that may be used to prepare pharmaceutically acceptable acid addition salts of axitinib are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. Axitinib may form pharmaceutically acceptable salts with various amino acids, in addition to the acids mentioned above.

The term “subject”, as used herein, may be a human or non-human mammal (e.g., rabbit, rat, mouse, horse, monkey, other lower-order primate, etc.).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

“Unit dosage form”, as used herein, refers to a physically discrete unit of inventive formulation appropriate for the subject to be treated. It will be understood, however, that the total daily usage of the compositions of the present invention will be decided by the attending physician within the scope of sound medical judgment. The specific effective dose level for any particular subject will depend upon a variety of factors including the disorder being treated and the severity of the disorder; specific composition employed; age, body weight, general health, sex and diet of the subject; time of administration, duration of the treatment; drugs and/or additional therapies used in combination or coincidental with the inventive compositions, and like factors well known in the medical arts.

The pharmaceutically acceptable composition or pharmaceutical composition of the present invention may be a solid pharmaceutical composition or formulation suitable for oral administration. The solid formulation may be a tablet or a capsule, such as a hard-shell capsule. In one embodiment, the tablet is a film coated tablet. The capsule may be a dry-filled or a microsphere-filled capsule.

The pharmaceutical composition comprises axitinib or a pharmaceutically acceptable salt thereof and excipients. In an embodiment, the axitinib has a mean particle size, which is acceptable for content uniformity. A suitable particle size for axitinib may be D(v, 0.5) NMT 25 microns or D(v, 0.9) NMT 81 microns. D(v, 0.5) NMT 25 microns means that means 50% of the particles are smaller than 25 microns and 50% are larger. D(v, 0.9) NMT 81 microns means that 90% of the particles are smaller than 81 microns and 10% are larger.

In an embodiment, the present invention relates to a photostable pharmaceutical composition comprising axitinib or a pharmaceutical salt thereof. In another embodiment, the present invention relates to a photostable pharmaceutical composition comprising axitinib and excipients, or a pharmaceutical salt thereof.

In another embodiment, the present invention relates to a photostable pharmaceutical composition comprising a core and a coating, the core comprising axitinib or a pharmaceutically acceptable salt thereof and excipients, and the coating comprising a metal oxide.

The pharmaceutical composition of the present invention includes a core and a coating. The core includes axitinib or a pharmaceutically acceptable salt thereof and excipients. The pharmaceutically acceptable core excipients may include fillers, disintegrants and lubricants.

Suitable fillers or diluents are known in the art. Suitable fillers include ductile fillers and brittle fillers. For example, suitable fillers include, but are not limited to, lactose (monohydrate, spray-dried monohydrate, anhydrous and the like), lactilol, starch, dextrin, glucose, silicic acid, sucrose, Sorbitol, Sodium Saccharin, Acesulfame potassium, Xylitol, Aspartame, Mannitol, polyvinyl pyrrolidone, low molecular weight hydroxypropyl cellulose, microcrystalline cellulose, silicified microcrystalline cellulose, low molecular weight hydroxypropyl methylcellulose, low molecular weight carboxymethyl cellulose, ethylcellulose, a suitable inorganic calcium salt such as dicalcium phosphate, alginates, gelatin, polyethylene oxide, acacia, magnesium aluminum silicate, and polymethacrylates, or a combination thereof. In one embodiment, fillers include agents selected from the group consisting of microcrystalline cellulose and lactose monohydrate, or a combination thereof. The filler comprises from about 87 weight percent to about 97 weight percent of the composition, based upon total weight of the composition. In an embodiment, the filler comprises from about 89 weight percent to about 97 weight percent of the composition, based upon total weight of the composition. In another embodiment, the filler comprises from about 92 weight percent to about 97 weight percent of the composition, based upon total weight of the composition. In another embodiment, the filler comprises from about 87 weight percent to about 95 weight percent of the composition, based upon total weight of the composition. In another embodiment, the filler comprises from about 90 weight percent to about 95 weight percent of the composition, based upon total weight of the composition.

Suitable disintegrants are also known in the art. Suitable disintegrants include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate, or a combination thereof. In one embodiment, the disintegrant includes croscarmellose sodium. The disintegrant comprises from about 2 weight percent to about 5 weight percent of the composition, based upon total weight of the composition. In an embodiment, the disintegrant comprises from about 2 weight percent to about 4 weight percent of the composition, based upon total weight of the composition.

Suitable lubricants are also known in the art. Suitable lubricants include, but are not limited to, magnesium stearate, calcium stearate, zinc stearate, sodium stearyl fumarate, and mixtures of magnesium stearate with sodium lauryl sulphate, or combinations thereof. In one embodiment, the lubricant includes magnesium stearate. The lubricant comprises from about 0.25 weight percent to about 5 weight percent of the composition by weight, based upon total weight of the composition. In an embodiment, the lubricant comprises from about 0.25 weight percent to about 3 weight percent of the composition by weight, based upon total weight of the composition.

A suitable coating or coating excipient of the present invention includes metal oxide. In an embodiment, the metal oxide coating or coating excipient includes iron oxide. The metal oxide, such as iron oxide, comprises from about 5 weight percent to about 20 weight percent of the coating by weight, based upon total weight of the coating formulation or composition. In an embodiment, the metal oxide, such as iron oxide, comprises about 7 weight percent, about 9 weight percent or about 18 weight percent of the coating by weight, based upon total weight of the coating composition. In an embodiment, the metal oxide, such as iron oxide, comprises about 7 weight percent, about 9.5 weight percent or about 17.5 weight percent of the coating by weight, based upon total weight of the coating composition. In an embodiment, the metal oxide, such as iron oxide, comprises from about 7 weight percent of the coating by weight, based upon total weight of the coating composition.

In an embodiment, the coating or coating excipients include a metal oxide, such as iron oxide, and may further include polymers, plasticizers, opacifiers, diluents or fillers, and colorants.

In an embodiment, the coating of the present invention is an aqueous coating. The coating or aqueous coating of the present invention comprises a polymer, a plasticizer, an opacifier, a pharmaceutically acceptable diluent or filler and optionally a colorant.

Suitable polymers are known in the art. Suitable polymers include, but are not limited to, cellulosics such as hydroxypropyl methylcellulose, hydroxypropylcellulose, hydroxyethylcellulose, methylhydroxyethylcellulose, methylcellulose, and sodium carboxymethylcellulose. Further examples of polymers include vinyls such as polyvinyl pyrrolidone. In an embodiment, the polymer is hydroxypropyl methylcellulose. The polymer comprises from about 25 weight percent to about 30 weight percent of the coating by weight, based upon total weight of the coating composition. In an embodiment, the polymer comprises about 28 weight percent of the coating by weight, based upon total weight of the coating composition.

Suitable plasticizers are known in the art. Suitable plasticizers include, but are not limited to, polyhydric alcohols such as glycerol and polyethylene glycols and acetate esters such as glycerol triacetate or glyceryl triacetate, which are known as triacetin, and triethyl citrate. In an embodiment, the plasticizer is triacetin. The plasticizer comprises from about 5 weight percent to about 10 weight percent of the coating by weight, based upon total weight of the coating composition. In an embodiment, the plasticizer comprises about 8 weight percent of the coating by weight, based upon total weight of the coating composition.

Suitable opacifiers are known in the art. Suitable opacifiers include, but are not limited to metal oxides, such as titanium dioxide or iron oxide, and talc. In an embodiment, the opacifier is titanium dioxide and iron oxide. In an embodiment, the opacifier is titanium dioxide. In an embodiment, the opacifier is iron oxide. The opacifier comprises from about 4 weight percent to about 25 weight percent of the coating by weight, based upon total weight of the coating composition. In an embodiment, the opacifier comprises from about 4 weight percent to about 20 weight percent of the coating by weight, based upon total weight of the coating composition. In an embodiment, the opacifier comprises about 24 weight percent of the coating by weight, based upon total weight of the coating composition. In an embodiment, the opacifier comprises about 6 weight percent, about 14 weight percent or about 17 weight percent of the coating by weight, based upon total weight of the coating composition. In an embodiment, the opacifier comprises from about 17 weight percent of the coating by weight, based upon total weight of the coating composition.

Suitable fillers or diluents are known in the art. Suitable fillers include ductile fillers and brittle fillers. For example, suitable fillers include, but are not limited to, lactose (monohydrate, spray-dried monohydrate, anhydrous and the like), lactilol, starch, dextrin, glucose, silicic acid, sucrose, Sorbitol, Sodium Saccharin, Acesulfame potassium, Xylitol, Aspartame, Mannitol, polyvinyl pyrrolidone, low molecular weight hydroxypropyl cellulose, microcrystalline cellulose, silicified microcrystalline cellulose, low molecular weight hydroxypropyl methylcellulose, low molecular weight carboxymethyl cellulose, ethylcellulose, a suitable inorganic calcium salt such as dicalcium phosphate, alginates, gelatin, polyethylene oxide, acacia, magnesium aluminum silicate, and polymethacrylates, or a combination thereof. In one embodiment, the filler is lactose monohydrate. The filler comprises about 40 weight percent of the coating by weight, based upon total weight of the coating composition.

Optionally, the compositions of the present invention may include a colorant or a glidant. Such colorants are available from a number of commercial vendors and are well known to those skilled in the art. In an embodiment, the colorant is a metal oxide, such as an iron oxide. Suitable glidants are known in the art. Suitable glidants include, but are not limited to, silicon dioxide, talc and cornstarch.

In certain embodiments, the coating for a film coated tablet includes a film coating system that contains a filler, a polymer, a plasticizer, an opacifier and pigmented iron oxide. A suitable film coating system is the Opadry® II Complete Film Coating System (Colorcon). In one embodiment, the coating is selected from the group consisting of Opadry® II Red, Opadry® II Yellow, and Opadry® II Gray. In another embodiment, the coating is Opadry® II Red.

The compositions of the Opadry® II Red, Opadry® II Yellow and Opadry® II Gray film coating systems are shown in Table 1 below.

TABLE 1
Opadry ® II Red, Opadry ® II Yellow and
Opadry ® II Gray Compositions

| | | | | Opadry ® |
|---|---|---|---|---|
| | | Opadry ® II | Opadry ® II | II Gray |
| | | Red | Yellow | (black |
| | | (red iron | (yellow iron | iron |
| | | oxide) | oxide) | oxide) |
| Component | Function | (w/w %) | (w/w %) | (w/w %) |
| Lactose | Brittle | 40.000 | 40.000 | 40.000 |
| Monohydrate | Filler | | | |
| HPMC 2910/ | Polymer | 28.000 | 28.000 | 28.000 |
| Hypromellose | | | | |
| 15 cP | | | | |
| Triacetin/Glycerol | Plasticizer | 8.000 | 8.000 | 8.000 |
| Triacetate | | | | |
| Titanium Dioxide | Opacifier | 17.090 | 6.384 | 14.500 |
| Iron Oxide | Photo- | 6.910 | 17.616 | 9.500 |
| | protection | | | |

The coating or coating excipients of the present invention comprise from about 1 weight percent to about 8 weight percent of the composition, based upon total weight of the composition. The coating or coating excipients of the present invention comprise from about 1 weight percent to about 9 weight percent of the composition, based upon total weight of the composition. In an embodiment, the coating of the present invention comprises from about 2 weight percent to about 5 weight percent of the composition, based upon total weight of the composition. In an embodiment, the coating of the present invention comprises from about 4 weight percent of the composition, based upon total weight of the composition.

In an embodiment, the composition of axitinib 1 mg Form XLI red film coated tablets is shown in Table 2 below.

TABLE 2
Composition of Axitinib 1 mg Form XLI Red Film Coated Tablets

| | | | | kg/ |
|---|---|---|---|---|
| Component | Function | mg/tablet | w/w % | batch |
| Axitinib Form XLI¹ | API | 1.000 | 1.000 | 0.750 |
| Microcrystalline Cellulose, | Ductile | 63.250 | 63.250 | 47.437 |
| grade 102¹ | Filler | | | |
| Lactose Monohydrate | Brittle Filler | 32.000 | 32.000 | 24.000 |
| Croscarmellose Sodium | Disintegrate | 3.000 | 3.000 | 2.250 |
| Magnesium Stearate² | Lubricant | 0.250 | 0.250 | 0.188 |
| Magnesium Stearate³ | Lubricant | 0.500 | 0.500 | 0.375 |
| Core Total (mgW) | | 100.000 | 100.000 | 75.000 |
| Opadry ® II Red⁴ | Coating | 4.000 | 4.000 | 3.000 |
| Purified Water⁵ | Solvent | N/A | N/A | 17.000 |
| Tablet Total (mgW) | | 104.000 | | 78.000 |

¹The exact amount of axitinib to be weighed will be adjusted for potency. The amount of microcrystalline cellulose will be adjusted accordingly.
²As vegetable grade, added at the blending step
³As vegetable grade, added at the final blending step
⁴The composition is provided in Table 1 above.
⁵Evaporated during processing and does not appear in the final product

In the embodiments of the compositions of the present invention, it will be appreciated that the exact amount of axitinib to be weighed will be adjusted for potency. The potency of axitinib, in a free base form or a pharmaceutical salt thereof, will be determined in order to calculate the exact weight of axitinib, in a free base form or a pharmaceutical salt thereof, which is required to reach the desired mg of the free base form of axitinib, in the composition.

In an embodiment, the composition of axitinib 3 mg Form XLI red film coated tablets is shown in Table 3 below.

TABLE 3
Composition of Axitinib 3 mg Form XLI Red Film Coated Tablets

| | | | | kg/ |
|---|---|---|---|---|
| Component | Function | mg/tablet | w/w % | batch |
| Axitinib Form XLI¹ | API | 3.000 | 2.857 | 2.143 |
| Microcrystalline Cellulose, | Ductile | 64.458 | 61.389 | 46.041 |
| grade 102¹ | Filler | | | |
| Lactose Monohydrate² | Brittle Filler | 33.600 | 32.000 | 24.000 |
| Croscarmellose Sodium | Disintegrate | 3.150 | 3.000 | 2.250 |
| Magnesium Stearate² | Lubricant | 0.264 | 0.251 | 0.189 |
| Magnesium Stearate³ | Lubricant | 0.528 | 0.503 | 0.377 |
| Core Total (mgW) | | 105.000 | 100.000 | 75.000 |
| Opadry ® II Red⁴ | Coating | 4.200 | 4.000 | 3.000 |
| Purified Water⁵ | Solvent | N/A | N/A | 17.000 |
| Tablet Total (mgW) | | 109.200 | | 78.000 |

¹The exact amount of axitinib to be weighed will be adjusted for potency. The amount of microcrystalline cellulose will be adjusted accordingly.
²As vegetable grade, added at the blending step
³As vegetable grade, added at the final blending step
⁴The composition is provided in Table 1 above.
⁵Evaporated during processing and does not appear in the final product

In an embodiment, the composition of axitinib 5 mg Form XLI red film coated tablets is shown in Table 4 below.

TABLE 4
Composition of Axitinib 5 mg Form XLI Red Film Coated Tablets

| | | | | kg/ |
|---|---|---|---|---|
| Component | Function | mg/tablet | w/w % | batch |
| Axitinib Form XLI¹ | API | 5.000 | 2.857 | 2.143 |
| Microcrystalline Cellulose, | Ductile | 107.430 | 61.389 | 46.041 |
| grade 102¹ | Filler | | | |
| Lactose Monohydrate² | Brittle Filler | 56.000 | 32.000 | 24.000 |
| Croscarmellose Sodium | Disintegrate | 5.250 | 3.000 | 2.250 |
| Magnesium Stearate² | Lubricant | 0.440 | 0.251 | 0.189 |
| Magnesium Stearate³ | Lubricant | 0.880 | 0.503 | 0.377 |
| Core Total (mgW) | | 175.000 | 100.000 | 75.000 |
| Opadry ® II Red⁴ | Coating | 7.000 | 4.000 | 3.000 |
| Purified Water⁵ | Solvent | N/A | N/A | 17.000 |
| Tablet Total (mgW) | | 182.000 | | 78.000 |

¹The exact amount of axitinib to be weighed will be adjusted for potency. The amount of microcrystalline cellulose will be adjusted accordingly.
²As vegetable grade, added at the blending step
³As vegetable grade, added at the final blending step
⁴The composition is provided in Table 1 above.
⁵Evaporated during processing and does not appear in the final product

In an embodiment, the composition of axitinib 7 mg Form XLI red film coated tablets is shown in Table 5 below.

TABLE 5
Composition of Axitinib 7 mg Form XLI Red Film Coated Tablets

| | | | | kg/ |
|---|---|---|---|---|
| Component | Function | mg/tablet | w/w % | batch |
| Axitinib Form XLI¹ | API | 7.000 | 2.857 | 1.000 |
| Microcrystalline Cellulose, | Ductile | 150.403 | 61.389 | 21.486 |
| grade 102¹ | Filler | | | |
| Lactose Monohydrate² | Brittle Filler | 78.400 | 32.000 | 11.200 |
| Croscarmellose Sodium | Disintegrate | 7.350 | 3.000 | 1.050 |
| Magnesium Stearate² | Lubricant | 0.616 | 0.251 | 0.088 |
| Magnesium Stearate³ | Lubricant | 1.231 | 0.503 | 0.176 |
| Core Total (mgW) | | 245.000 | 100.000 | 35.000 |
| Opadry ® II Red⁴ | Coating | 9.800 | 4.000 | 1.400 |
| Purified Water⁵ | Solvent | N/A | N/A | 7.933 |
| Tablet Total (mgW) | | 254.800 | | 36.400 |

¹The exact amount of axitinib to be weighed will be adjusted for potency. The amount of microcrystalline cellulose will be adjusted accordingly.
²As vegetable grade, added at the blending step
³As vegetable grade, added at the final blending step
⁴The composition is provided in Table 1 above.
⁵Evaporated during processing and does not appear in the final product

The pharmaceutical composition or solid formulation of the present invention may be manufactured by a conventional dry granulation, direct compression, wet granulation, drug layering or liquid-filled manufacturing process using equipment commonly available in the pharmaceutical industry.

In an embodiment, the pharmaceutical composition or solid formulation of the present invention may be manufactured by a conventional dry granulation manufacturing process that includes blending, milling, blend lubrication, roller compaction and milling, blend lubrication, compression, and aqueous based film coating using equipment commonly available in the pharmaceutical industry.

In an embodiment, the pharmaceutical composition of the present invention may be manufactured using the process described below.

### Blend and Dry Granulate

Step 1. Charge the microcrystalline cellulose, axitinib, croscarmellose sodium and lactose monohydrate into a suitable diffusion mixer and blend.
Step 2. Mill the blend from Step 1 through a suitable screening mill into a suitable diffusion mixer.
Optional Step 3. Optionally charge blend from Step 2 into a suitable diffusion mixer then blend.
Step 4. Charge magnesium stearate (approximately one third) into the suitable diffusion mixer from Step 2 or Step 3 then blend.
Step 5. Dry granulate the blend from Step 4 using a dry granulator.
Step 6. Mill the compacted blend from Step 5 using a suitable screening mill into a suitable diffusion mixer.
Optional Step 7. Optionally charge blend from Step 6 into a suitable diffusion mixer then blend.
Step 8. Charge magnesium stearate (approximately two-thirds) into the suitable diffusion mixer from Step 6 then blend.

### Preparation of Core Tablet

Step 9. Compress the granulated blend from Step 8 on a tablet press and compress into core tablets.

### Preparation of Film Coated Tablet

Step 10. Add purified water to a vessel. While mixing the contents with a propeller mixer, add a suitable coating excipient and mix until the solids are well dispersed and free of lumps.
Optional Step 11. Add purified water to a vessel. While mixing the contents with a propeller mixer, add the Opadry® Clear (YS-2-19114-A) and mix until the solids are completely dissolved.
Step 12. Charge a suitable pan load of tablet cores from Step 9 into a suitable pan coater.
Step 13. With the coating pan rotating at an appropriate speed, apply the coating suspension from Step 10 until the appropriate level of coating is achieved.
Optional Step 14. With the coating pan rotating at an appropriate speed, apply the coating suspension from optional Step 11 until the appropriate level of coating is achieved.

In an embodiment, the axitinib 1 mg form XLI red film coated tablets are prepared according to the procedure described below.
Step 1. Charge the microcrystalline cellulose, axitinib, croscarmellose sodium and lactose monohydrate into a suitable diffusion mixer and blend.
Step 2. Mill the blend from Step 1 through a suitable screening mill into a diffusion mixer.
Step 3. Charge magnesium stearate (approximately one third) into the diffusion mixer from Step 2 then blend.
Step 4. Dry granulate the blend from Step 3 using a dry granulator.
Step 5. Mill the compacted blend from Step 4 using a suitable screening mill into a diffusion mixer.
Step 6. Charge magnesium stearate (approximately two-thirds) into the diffusion mixer from Step 5 then blend.
Step 7. Compress the granulated blend from Step 6 on a tablet press and compress into tablets.
Step 8. Add Purified Water to a vessel. While mixing the contents with a propeller mixer, add Opadry® II Red and mix until the solids are well dispersed and free of lumps.
Step 9. Charge a suitable pan load of tablet cores from Step 7 into a suitable pan coater.
Step 10. With the coating pan rotating at an appropriate speed, apply the coating suspension from Step 8 until the appropriate level of coating is achieved.

In an embodiment, the axitinib 3 mg, 5 mg and 7 mg Form XLI red film coated tablets are prepared according to the procedure described immediately above for the axitinib 1 mg Form XLI red film coated tablets, with the exception that the 5 mg tablets are film coated in two portions.

Alternatively, the active pharmaceutical ingredient and excipients of the present invention may be filled into hard-shell capsules, also referred to as the dry-filled capsules or microsphere-filled capsules. The capsule formulation and manufacturing process are similar to the tablet core formulation and manufacturing process. A hard-shell capsule may consist of gelatin and water or hydroxypropyl methylcellulose, water and a gelling agent (gelan gum or carageenan). Such capsule compositions do not utilize an aqueous coating. The encapsulated pharmaceutical composition comprises about 2.0 weight percent to about 10 weight percent of a disintegrant, about 0.1 weight percent to about 0.5 weight percent of a glidant, about 0.25 weight percent to about 5.0 weight percent of a lubricant and about 81.0 weight percent to about 96 weight percent of a diluent or filler.

The pharmaceutical compositions of the present invention may be formulated into a unit dosage form. Such formulations are well known to one of ordinary skill in the art. In an embodiment, the present invention provides a pharmaceutical composition comprising a solid unit dosage form as a tablet. In other embodiments, the present invention provides a pharmaceutical composition comprising a unit dosage form as a microsphere or dry-filled capsule. In some embodiments, a unit dosage form contains 1 mg, 3 mg, 5 mg, 7 mg or 10 mg of axitinib. In some embodiments, a unit dosage form contains 1 mg, 5 mg, or 10 mg of axitinib. In some embodiments, a unit dosage form contains 1 mg or 5 mg of axitinib. In some embodiments, a unit dosage form comprises a tablet that contains 1 mg or 5 mg of axitinib. In some embodiments, a unit dosage form contains between 1 mg and 10 mg, inclusive, of axitinib.

In some embodiments, satisfactory results are obtained when axitinib, or a pharmaceutically acceptable salt thereof, is administered at a daily dosage of from about 1 mg to about 25 mg, optionally given in divided doses two times a day. The total daily dosage is projected to be from about 1 mg to about 10 mg two times a day, preferably from about 5 to about 10 mg two times a day. This dosage regimen may be adjusted to provide the optimal therapeutic response. For example, several divided doses may be administered daily or the dose may be proportionally reduced or increased as indicated by the exigencies of the therapeutic situation.

An embodiment relates to methods of treating abnormal cell growth in a subject comprising administering to the subject an amount of a pharmaceutical composition according to the present invention. In an embodiment, the abnormal cell growth is cancer. In another embodiment, the cancer is liver cancer, melanoma, mesothelioma, non-small cell lung cancer, prostate cancer, renal cell carcinoma, soft tissue sarcomas and solid tumors.

The pharmaceutical composition of the present invention provides protection of axitinib from degradation, including photodegradation and oxidative degradation. In an embodiment, the pharmaceutical composition of the present invention provides protection of axitinib from photodegradation.

The pharmaceutical composition of the present invention provides protection of axitinib from degradation throughout prolonged storage. Prolonged storage may be at least 9 months, at least 12 months, at least 24 months or at least 36 months. In an embodiment, prolonged storage may be at least 36 months.

Degradation products of the pharmaceutical composition of the present invention include photodegradants and oxidative degradants. The photogradants include compounds of Formula I, Formula II and Formula III, shown below. The compound of Formula I may be referred to as the 2+2 dimer, the compound of Formula II may be referred to as the asymmetric dimer, and the compound of Formula III may be referred to as the cis-isomer. The oxidative degradants include the compound of Formula IV, which may be referred to as the sulfoxide derivative.

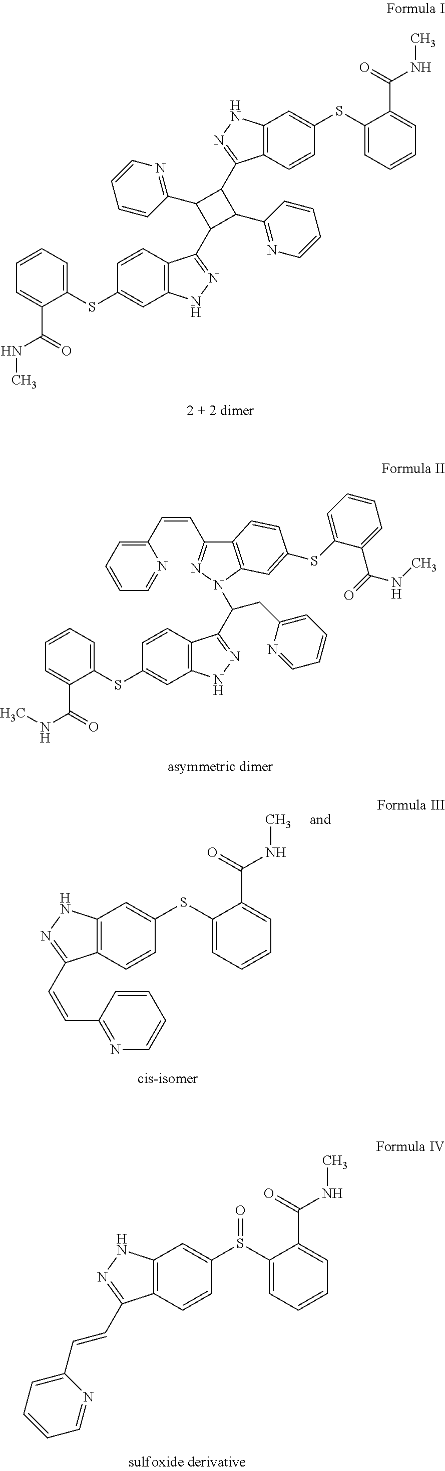

The 2+2 dimer and the cis-isomer are the major photodegradants of the pharmaceutical composition comprising axitinib Form IV and Form XXV. The asymmetric dimer and the cis-isomer are the major photodegradants of the pharmaceutical composition comprising axitinib Form XLI.

HPLC, SFC, TLC are techniques that may be used to detect degradation products, including photodegradants and oxidative degradants.

An example of a suitable HPLC assay is gradient elution reversed-phase liquid chromatography, which may be used to separate axitinib from degradation products and formulation excipients. Comparison of the peak area response and retention time of axitinib for a sample and the standard provides a quantitative assay and identification test for axitinib. Degradation products of the present invention are identified by their retention time relative to axitinib and quantitated by area percent.

The assay may be conducted with equipment, methodology and reagents well known in the art. For example, the assay may utilize a suitable liquid chromatograph. The suitable liquid chromatograph may include a pump, constant flow delivery, an ultraviolet (UV) detector, an injector or autosampler, and/or a column heater. The suitable liquid chromatograph may include a UV detector capable of operating at between about 205 nm and about 400 nm, an injector or autosampler capable of making about 1 to about 100 microliter injections, and/or a column heater capable of maintaining temperature of 25° C. The suitable liquid chromatograph may also include an integrator/data acquisition system. The assay may utilize a HPLC column. A suitable column is a Waters Symmetry C₁₈, 5 micron 4.6 mm ID×150 mm length column. The assay may utilize sample filters. A suitable sample filter is an Acrodisc® CR 25 mm syringe filter with 0.45 μm PTFE membrane (PALL Life Sciences, part number 4219T). The assay may utilize an analytical balance. A suitable analytical balance may be capable of measurements to ±0.01 mg. The assay may utilize an ultrasonic bath. A suitable ultrasonic bath is a Bransonic Ultrasonic Cleaner 3210R-MT. The assay may utilize a reciprocating mechanical shaker. A suitable reciprocating mechanical shaker is an IKA Labortechnik HS501 shaker. The assay may also utilize amber volumetric glassware and autosampler vials.

It is known that axitinib, as an active pharmaceutical ingredient, can exist in multiple crystalline or polymorphic forms. The crystalline forms of axitinib include Form IV, Form XXV and Form XLI. Crystalline Form IV of axitinib API is described in U.S. Publication No. 2006-0094763. Crystalline Forms XXV and XLI of axitinib API are described in U.S. Publication No. 2010-0179329. These forms may be formulated in a drug product, such as the pharmaceutical composition of the present invention. Each crystalline form may have advantages over the other forms in terms of properties such as bioavailability, stability, and manufacturability.

The pharmaceutical composition of the present invention contains an API, axitinib, or a pharmaceutically acceptable salt thereof. Each crystalline form of axitinib, as formulated in the pharmaceutical composition or drug product of the present invention, can be characterized by one or more of the following: powder X-ray diffraction pattern (i.e., X-ray diffraction peaks at various diffraction angles (2θ), solid state nuclear magnetic resonance (NMR) spectrum, Raman spectrum, aqueous solubility, light stability under ICH high intensity light conditions, and physical and chemical storage stability.

Polymorphic Forms IV, XXV, and XLI, of axitinib within the drug product or pharmaceutical composition of the present invention were each characterized by the positions of peaks in their powder X-ray diffraction patterns. The powder X-ray diffraction patterns differ for each of the polymorphic forms of formulated axitinib. For example, Forms IV, XXV, and XLI of axitinib in drug product can be distinguished from each other and from other polymorphic forms of formulated axitinib by using powder X-ray diffraction. The detection of characteristic powder X-ray diffraction peaks of axitinib within the drug product or pharmaceutical composition of the present invention enables unique identification of polymorphic Forms IV, XXV, and XLI, of axitinib in the drug product or pharmaceutical composition.

The powder X-ray diffraction patterns of the axitinib pharmaceutical compositions were generated using a Siemens D5000 diffractometer using copper radiation (Cu K_{α1}, wavelength: 1.54056 Å). The instrument was equipped with a line focus X-ray tube. The tube voltage and amperage were set to 38 kV and 38 mA, respectively. The divergence and scattering slits were set at 1 mm, and the receiving slit was set at 0.6 mm. Diffracted Cu K_{α1} radiation was detected by a Sol-X energy dispersive X-ray detector. Tablets were prepared for analysis by light grinding in a small agate mortar and pestle. The powder samples were then placed in a quartz holder. A theta-two theta continuous scan at 0.2 degrees 2θ/minute (12 second/0.04 degrees 2θ step) from 3.0 to 40 degrees 2θ was used. Data were collected and analyzed using BRUKER AXS DIFFRAC PLUS software Version 2.0. An alumina standard was analyzed to check the instrument alignment. Samples were prepared by placing them in a quartz holder. It should be noted that Bruker Instruments purchased Siemans; thus, the Bruker D5000 instrument is essentially the same as a Siemans D5000. Eva Application 9.0.0.2 software was used to visualize and evaluate PXRD spectra. Generally, a Threshold value of 1 and a Width value of 0.3 were used to make preliminary peak assignments. The output of automated assignments was visually checked to ensure validity and adjustments were manually made if necessary. Additionally, peaks were manually assigned within spectra if appropriate. The characteristic peak values for polymorphic Forms IV, XXV, and XLI, of axitinib in the drug product or pharmaceutical composition are summarized in Tables 4, 5, and 6 below.

To perform an X-ray diffraction measurement on a Bragg-Brentano instrument like the Bruker system used for measurements reported herein, the sample is typically placed into a holder which has a cavity. The sample powder is pressed by a glass slide or equivalent to ensure a random surface and proper sample height. The sample holder is then placed into the instrument. The incident X-ray beam is directed at the sample, initially at a small angle relative to the plane of the holder, and then moved through an arc that continuously increases the angle between the incident beam and the plane of the holder.

Measurement differences associated with such X-ray powder analyses result from a variety of factors including: (a) errors in sample preparation (e.g., sample height); (b) instrument errors (e.g., flat sample errors); (c) calibration errors; (d) operator errors (including those errors present when determining the peak locations); and (e) the nature of the material (e.g., preferred orientation and transparency errors). Calibration errors and sample height errors often result in a shift of all the peaks in the same direction. Small differences in sample height when using a flat holder will lead to large displacements in PXRD peak positions. A systematic study showed that, using a Shimadzu XRD-6000 in the typical Bragg-Brentano configuration, sample height difference of 1 mm led to peak shifts as high as 1 degree 2θ (Chen et al., J Pharmaceutical and Biomedical Analysis 26:63 (2001)). These shifts can be identified from the X-ray diffractogram and can be eliminated by compensating for the shift (applying a systematic correction factor to all peak position values) or recalibrating the instrument. As mentioned above, it is possible to rectify measurements from the various machines by applying a systematic correction factor to bring the peak positions into agreement. In general, this correction factor will bring the measured peak positions from the Bruker into agreement with the expected peak positions and may be in the range of 0 to 0.2 degrees 2θ.

One of skill in the art will appreciate that the peak positions (2θ) will show some inter-apparatus variability, typically about 0.1 degrees 2θ. Accordingly, where peak positions (2θ) are reported, one of skill in the art will recognize that such numbers are intended to encompass such inter-apparatus variability. Furthermore, where the crystalline forms of the present invention are described as having a powder X-ray diffraction pattern essentially the same as that shown in a given figure, the term “essentially the same” is also intended to encompass such inter-apparatus variability in diffraction peak positions.

The intensities of the reflections within a powder X-ray diffraction peak list are typically expressed in relation to the largest intensity reflection within the sample spectrum. One skilled in the art will appreciate that relative peak intensities of the reflections within an API PXRD peak list will show inter-apparatus variability as well as variability due to a number of factors such as preferred orientation effects of crystals in the X-ray beam, the purity of the material being analyzed, or the degree of crystallinity of the sample. The relative intensities of the API reflections within a drug product sample may vary due to the factors mentioned above as well as additional factors brought about as a result of formulation. Since the majority of a drug product formulation typically consists of excipients the preferred orientation effects of excipient crystals in the X-ray beam, the purity of the crystalline excipient materials within the drug product sample, the degree of crystallinity of the excipients within the drug product sample, the loading of each excipient within the drug product, and the API loading within the drug product may also cause the relative intensities of reflections to vary within a drug product PXRD peak list.

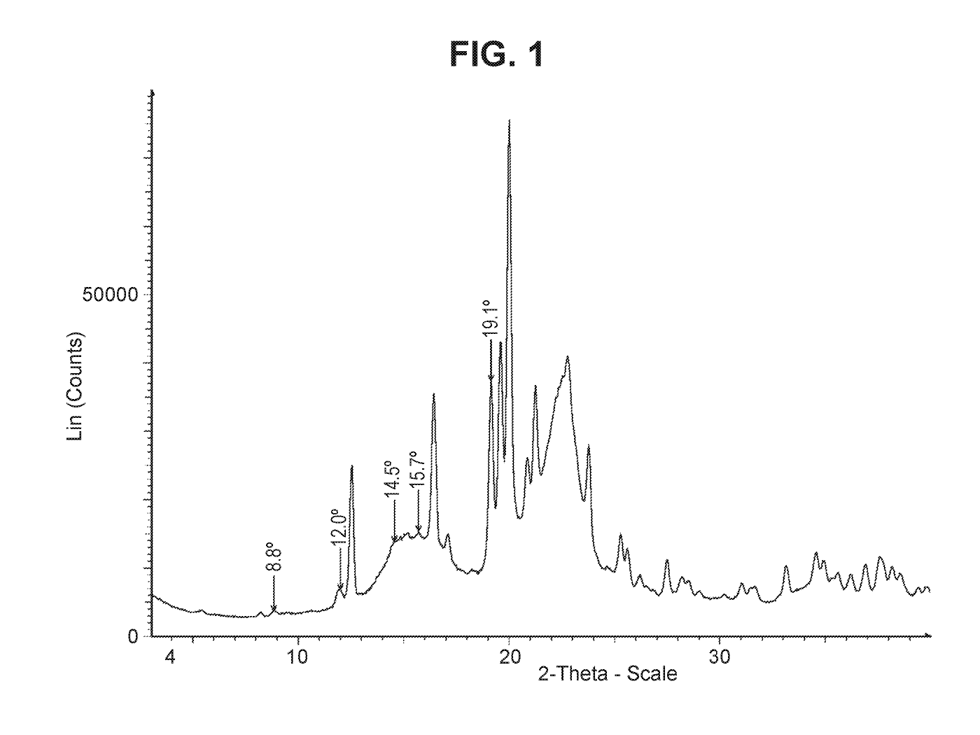

Crystalline Form IV of axitinib in drug product, which was prepared as provided in Example 8, was characterized by the PXRD pattern shown in FIG. 1. The PXRD pattern expressed in terms of the degree 2θ and relative intensities is shown in Table 6.

TABLE 6

| Angle | |
|---|---|
| (Degree 2θ) | Relative Intensity |
| 8.8 | 5 |
| 12.0 | 9 |
| 14.5 | 18 |
| 15.7 | 20 |
| 19.1 | 50 |

Form IV axitinib in the pharmaceutical composition of the present invention may be identified by a powder X-ray diffraction pattern comprising any one or more of the following 2θ values measured using CuKₐ radiation (λ=1.54056 Å): 8.8±0.1, 12.0±0.1, 14.5±0.1, 15.7±0.1 and 19.1±0.1.

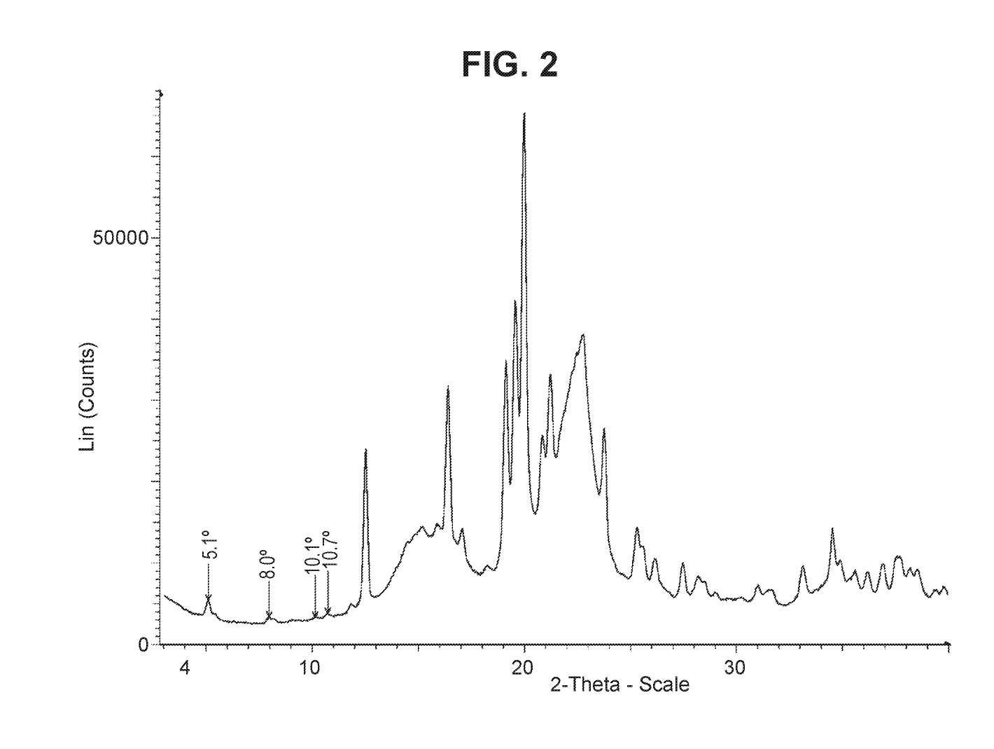

Crystalline Form XXV of axitinib in drug product, which was prepared as provided in Example 8, was characterized by the PXRD pattern shown in FIG. 2. The PXRD pattern expressed in terms of the degree 2θ and relative intensities is shown in Table 7.

TABLE 7

| Angle | |
|---|---|
| (Degree 2θ) | Relative Intensity |
| 5.1 | 8 |
| 8.0 | 5 |
| 10.1 | 5 |
| 10.7 | 6 |

Form XXV axitinib in the pharmaceutical composition of the present invention may be identified by a powder X-ray diffraction pattern comprising any one or more of the following 2θ values measured using CuKₐ radiation (λ=1.54056 Å): 5.1±0.1, 8.0±0.1, 10.1±0.1 and 10.7±0.1.

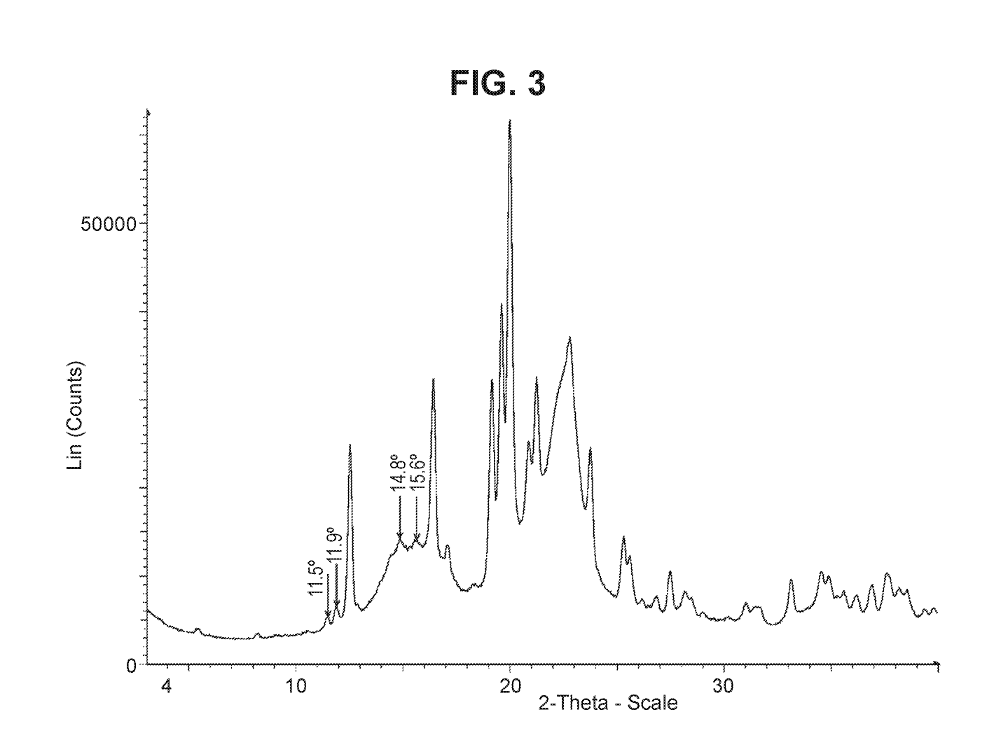

Crystalline Form XLI of axitinib in drug product, which was prepared as provided in Example 8, was characterized by the PXRD pattern shown in FIG. 3. The PXRD pattern expressed in terms of the degree 2θ and relative intensities is shown in Table 8.

TABLE 8

| Angle | |
|---|---|
| (Degree 2θ) | Relative Intensity |
| 11.5 | 9 |
| 11.9 | 11 |
| 14.8 | 23 |
| 15.6 | 23 |

Form XLI axitinib in the pharmaceutical composition of the present invention may be identified by a powder X-ray diffraction pattern comprising any one or more of the following 2θ values measured using CuKₐ radiation (λ=1.54056 Å): 11.5±0.1, 11.9±0.1, 14.8±0.1 and 15.6±0.1.

Polymorphic Form IV of axitinib API and polymorphic Forms IV, XXV, and XLI, of axitinib within the drug product or pharmaceutical composition of the present invention were each characterized using ¹³C SSNMR spectroscopy. The ¹³C solid state spectra differ for each of the polymorphic forms of formulated axitinib. For example, Forms IV, XXV, and XLI of axitinib in drug product can be distinguished from each other and from other polymorphic forms of formulated axitinib by using ¹³C SSNMR. The detection of characteristic ¹³C solid state spectra of axitinib within the drug product or pharmaceutical composition of the present invention enables unique identification of polymorphic Forms IV, XXV, and XLI, of axitinib in the drug product or pharmaceutical composition.

The ¹³C solid state spectra of Form IV of axitinib API were collected as follows. Approximately 80 mg of sample were tightly packed into a 4 mm ZrO₂ rotor. Spectra were collected at ambient temperature and pressure on a Bruker-Biospin 4 mm CPMAS probe positioned into a wide-bore Bruker-Biospin DSX 500 MHz (¹H frequency) NMR spectrometer. The packed rotor was oriented at the magic angle and spun at 15.0 kHz. The ¹³C solid state spectrum was collected using a proton decoupled cross-polarization magic angle spinning (CPMAS) experiment. The cross-polarization contact time was set to 2.0 ms. A proton decoupling field of approximately 90 kHz was applied. 550 scans were collected with a 30 second recycle delay. The carbon spectrum was referenced using an external standard of crystalline adamantane, setting its upfield resonance to 29.5 ppm.

The ¹³C solid state spectra of Forms IV, XXV, and XLI, of axitinib within the drug product or pharmaceutical composition of the present invention were collected as follows. Film coated tablets of the present invention were gently ground with a mortal and pestle. Approximately 300 mg of ground sample were tightly packed into a 7 mm ZrO₂ rotor. Spectra were collected at ambient temperature and pressure on a Bruker-Biospin 7 mm cross-polarization magic angle spinning (CPMAS) probe positioned into a wide-bore Bruker-Biospin DSX 500 MHz (¹H frequency) NMR spectrometer. The packed rotors were oriented at the magic angle and spun at 7.0 kHz. The ¹³C solid state spectra were collected using a proton decoupled CPMAS experiment with total suppression of spinning side bands (TOSS). The cross-polarization contact time was set to 2.0 ms. A proton decoupling field of approximately 76 kHz was applied. The spectrum of the axitinib Form IV formulation was acquired for 6,800 scans with a 22.5 second recycle delay. The spectrum of the axitinib Form XXV formulation was acquired for 1,536 scans with a 110 second recycle delay. The spectrum of the Axitinib Form XLI formulation was acquired for 768 scans with a 220 second recycle delay. Recycle delays were adjusted to approximately 1.25 times the proton longitudinal relaxation time of the corresponding API reference. Carbon spectra were referenced using an external standard of crystalline adamantane, setting its upfield resonance to 29.5 ppm.

Automatic peak picking was performed using Bruker-BioSpin TopSpin version 2.1 software. The peak picking regions were defined to exclude excipient resonances. The output of the automated peak picking was visually checked to ensure validity and adjustments manually made if necessary. Spinning side band intensities not suppressed in the ¹³C CPMAS TOSS experiment were manually removed from the peak lists.

The intensities of the chemical shifts within a CPMAS carbon spectrum can be expressed as peak heights in relation to the largest intensity chemical shift within the sample spectrum. As will be appreciated by the skilled person, the relative intensities of the chemical shifts within an active pharmaceutical ingredient solid-state NMR peak list may vary due to a number of factors such as the actual setup of the CPMAS experimental parameters, the thermal history of the sample, the purity of the material being analyzed, and the degree of crystallinity of the sample. The relative intensities of the active pharmaceutical ingredient chemical shifts within a drug product sample may vary due to the factors mentioned above as well as additional factors brought about as a result of formulation. The skilled person will also appreciate that CPMAS intensitied are not necessarily quantitative. Since the majority of a drug product formulation typically consists of excipients the purity of the excipient materials within the drug product sample, the degree of crystallinity of the excipients within the drug product sample, the loading of each excipient within the drug product, and the active pharmaceutical ingredient loading within the drug product may also cause the relative intensities of chemical shifts to vary within a drug product solid-state NMR peak list.

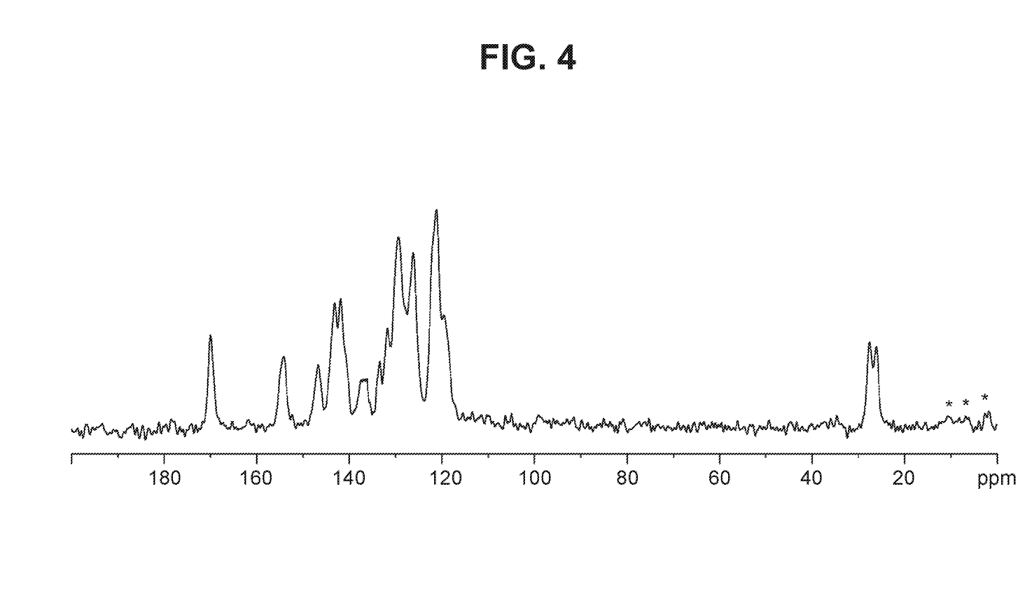

Crystalline Form IV of axitinib API was characterized by the solid state NMR spectrum shown in FIG. 4. The ¹³C chemical shifts of crystalline Form IV of axitinib API are shown in Table 9.

TABLE 9

| ¹³C Chemical Shifts | |
|---|---|
| [ppm] | Relative Intensity |
| 170.0 | 46 |
| 154.3 | 34 |
| 146.8 | 31 |
| 143.2 | 60 |
| 142.0 | 61 |
| 136.9 | 23 |
| 133.5 | 33 |
| 131.9 | 48 |
| 129.5 | 88 |
| 126.2 | 80 |
| 121.2 | 100 |
| 119.6 | 46 |
| 27.7 | 41 |
| 26.1 | 36 |

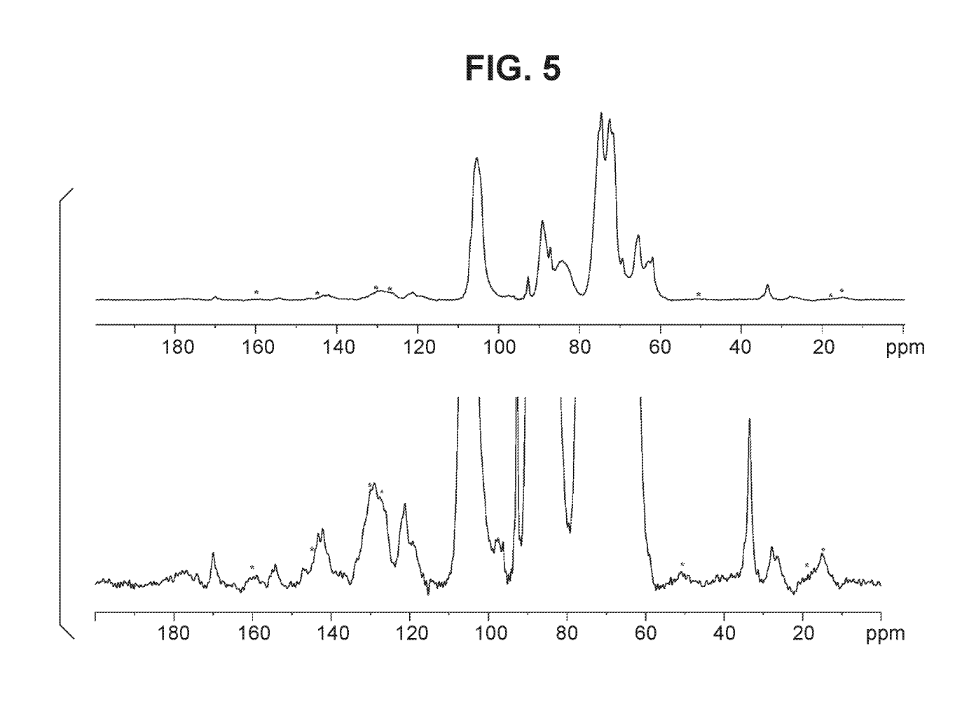

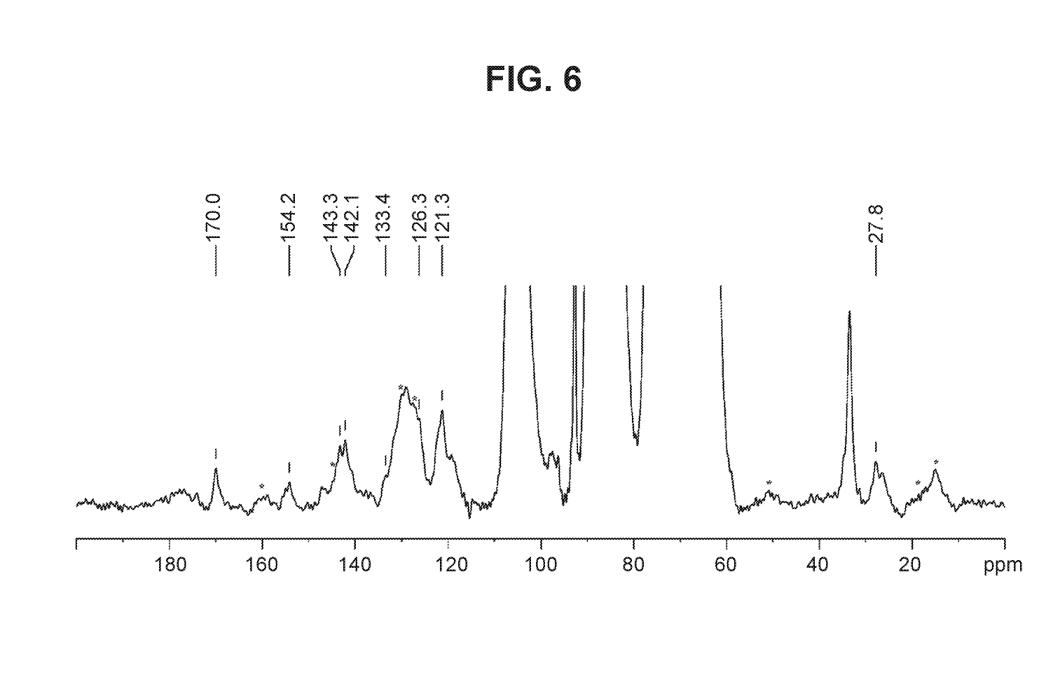

Crystalline Form IV of axitinib in drug product, which was prepared as provided in Example 8, as characterized by the solid state NMR spectrum shown in FIGS. 5 and 6. The ¹³C chemical shifts of crystalline Form IV of axitinib in drug product are shown in Table 10.

TABLE 10

| ¹³C Chemical Shifts | |
|---|---|
| [ppm] | Relative Intensity |
| 170.0 | 1 |
| 154.2 | 1 |
| 143.3 | 2 |
| 142.1 | 3 |
| 133.4 | 1 |
| 126.3^{a} | 4 |
| 121.3 | 4 |
| 27.8 | 2 |

^{a}Peak shoulder.

Form IV axitinib in the pharmaceutical composition of the present invention may be identified by a solid state nuclear magnetic resonance comprising any one or more of the following ¹³C chemical shifts expressed in parts per million: 170.0±0.2, 154.2±0.2, 143.3±0.2, 142.1±0.2, 133.4±0.2, 126.3±0.2, 121.3±0.2 and 27.8±0.2.

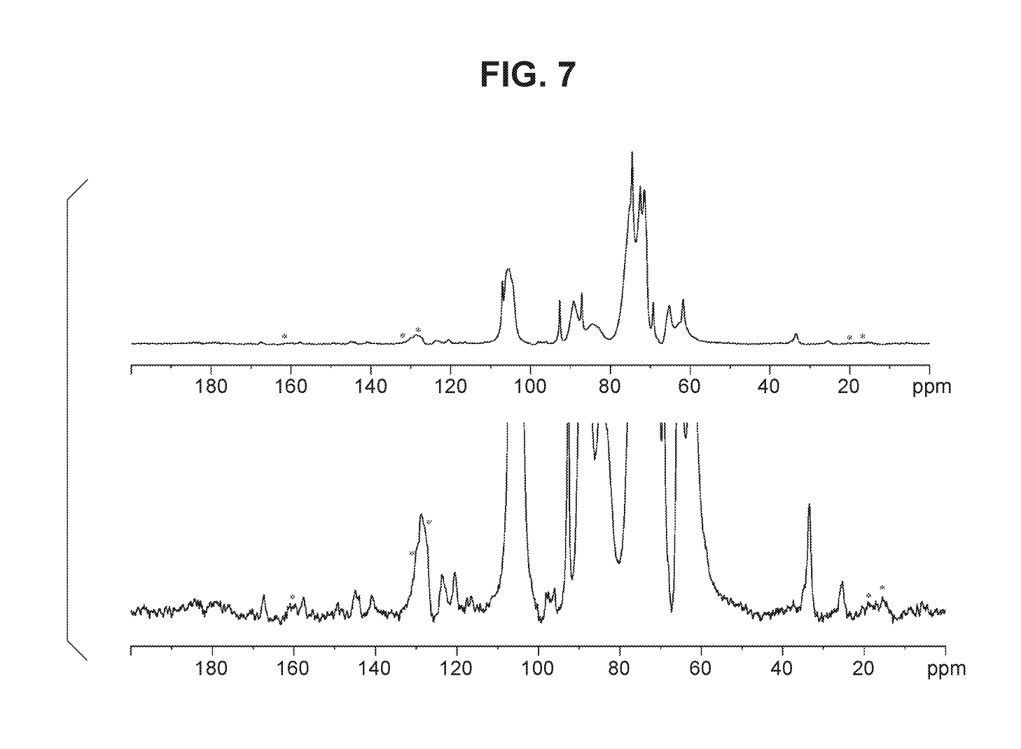

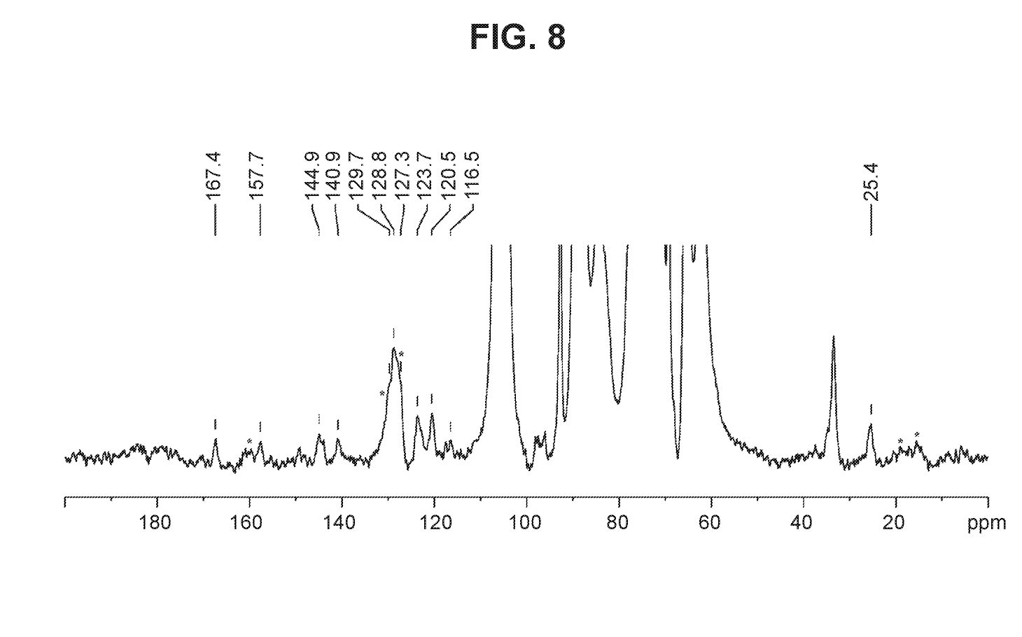

Crystalline Form XXV of axitinib in drug product, which was prepared as provided in Example 8, was characterized by the solid state NMR spectrum shown in FIGS. 7 and 8. The ¹³C chemical shifts of crystalline Form XXV of axitinib in drug product are shown in Table 11.

TABLE 11

| ¹³C Chemical Shifts | |
|---|---|
| [ppm] | Relative Intensity |
| 167.4 | 1 |
| 157.7 | 1 |
| 144.9 | 1 |
| 140.9 | 1 |
| 129.7^{a} | 3 |
| 128.8 | 5 |
| 127.3^{a} | 2 |
| 123.7 | 2 |
| 120.5 | 2 |
| 116.5 | 1 |
| 25.4 | 1 |

^{a}Peak shoulder

Form XXV axitinib in the pharmaceutical composition of the present invention may be identified by a solid state nuclear magnetic resonance comprising any one or more of the following ¹³C chemical shifts expressed in parts per million: 167.4±0.2, 157.7±0.2, 144.9±0.2, 140.9±0.2, 129.7±0.2, 128.8±0.2, 127.3±0.2, 123.7±0.2, 120.5±0.2, 116.5±0.2 and 25.4±0.2.

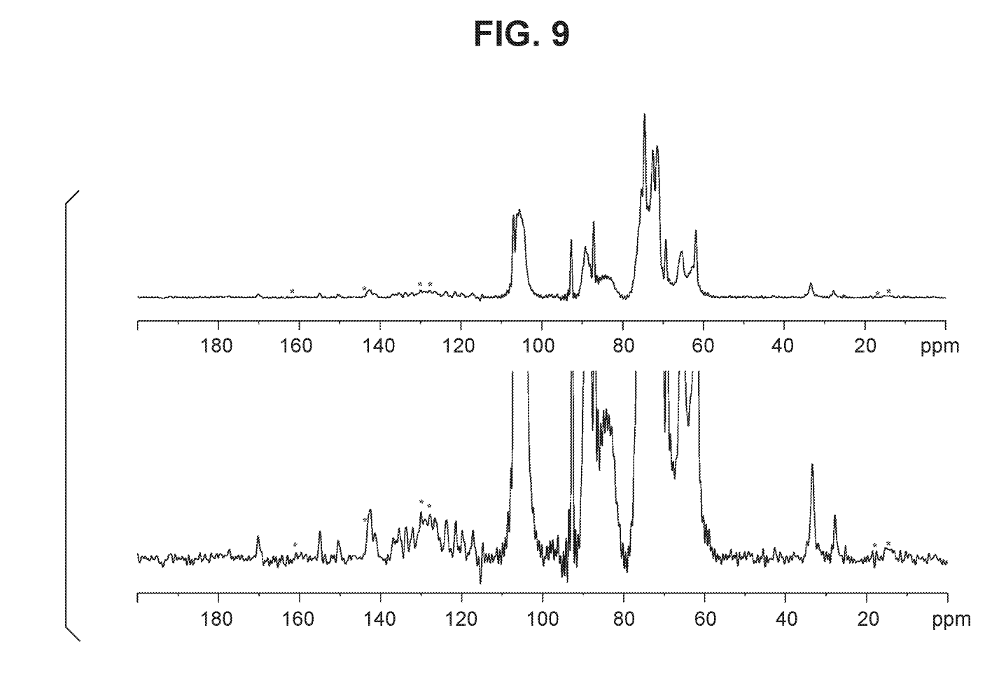

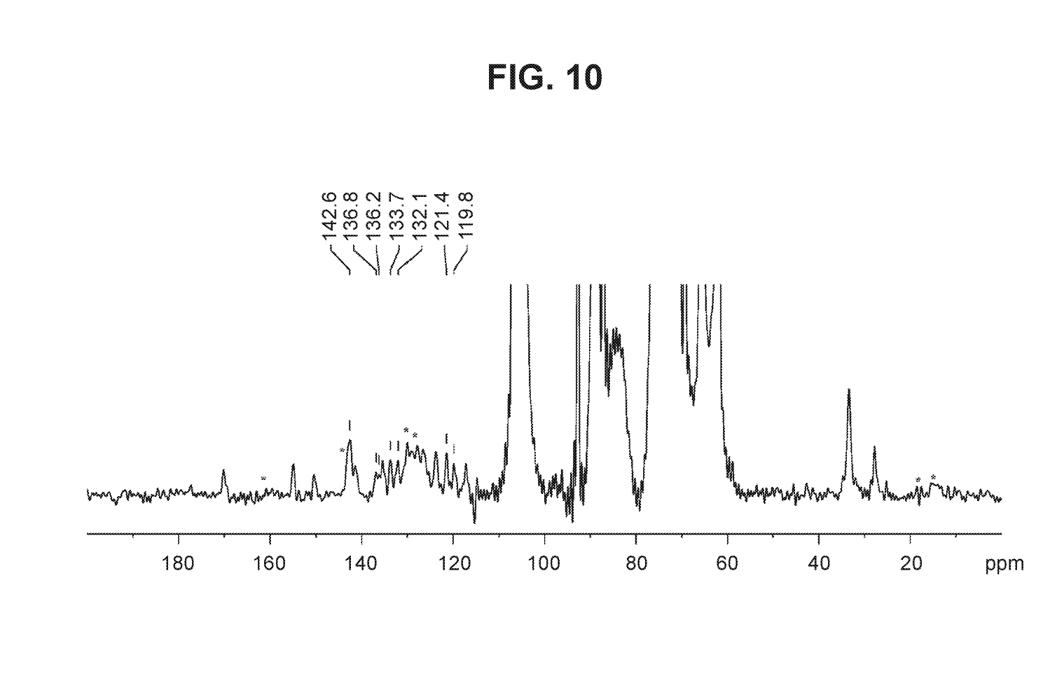

Crystalline Form XLI of axitinib in drug product, which was prepared as provided in Example 8, was characterized by the solid state NMR spectrum shown in FIGS. 9 and 10. The ¹³C chemical shifts of crystalline Form XLI of axitinib in drug product are shown in Table 12.

TABLE 12

| ¹³C Chemical Shifts | |
|---|---|
| [ppm]^{a} | Relative Intensity^{b} |
| 142.6 | 4 |
| 136.8 | 2 |
| 136.2 | 2 |
| 133.7 | 3 |
| 132.1 | 2 |
| 121.4 | 3 |
| 119.8 | 2 |

Form XLI axitinib in the pharmaceutical composition of the present invention may be identified by a solid state nuclear magnetic resonance comprising any one or more of the following ¹³C chemical shifts expressed in parts per million: 142.6±0.2, 136.8±0.2, 136.2±0.2, 133.7±0.2, 132.1±0.2, 121.4±0.2 and 119.8±0.2.

Polymorphic Form IV of axitinib API and polymorphic Forms IV, XXV, and XLI, of axitinib within the drug product or pharmaceutical composition of the present invention were each characterized using Raman spectroscopy. The Raman spectra differ for each of the polymorphic forms of formulated axitinib. For example, Forms IV, XXV, and XLI of axitinib in drug product can be distinguished from each other and from other polymorphic forms of formulated axitinib by using Raman spectroscopy. The detection of characteristic Raman spectra of axitinib within the drug product or pharmaceutical composition of the present invention enables unique identification of polymorphic Forms IV, XXV, and XLI, of axitinib in the drug product or pharmaceutical composition.

Raman spectra of Form IV of axitinib API were collected using a Nicolet NXR FT-Raman accessory attached to a Nicolet 6700 FTIR spectrometer equipped with a KBr beamsplitter and a d-TGS KBr detector. The spectrometer is equipped with a 1064 nm Nd:YVO₄ laser and a liquid nitrogen cooled Germanium detector. Prior to data acquisition, instrument performance and calibration verifications were conducted using polystyrene. Samples were analyzed in glass NMR tubes that were spun during spectral collection. The spectra were collected using 0.5 W of laser power and 400 co-added scans. The collection range was 3700-300 cm⁻¹. The API spectra were recorded using 2 cm⁻¹ resolution, and Happ-Genzel apodization was utilized for all of the spectra. A single spectrum was recorded for each sample, which was intensity normalized prior to peak picking.

Peaks were manually identified using the Thermo Nicolet Omnic 7.3a software. Peak position was picked at the peak maximum, and peaks were only identified as such, if there was a slope on each side; shoulders on peaks were not included. Both peak position and relative intensity values are reported in the peak tables for the neat API. The peak position has been rounded to the nearest whole number using standard practice (0.5 rounds up, 0.4 rounds down). The relative intensity values were grouped into strong (S), medium (M) and weak (W) for the neat API using the following divisions: strong (1-0.75); medium (0.74-0.3) and weak (0.29 and below).

Raman spectra of Forms IV, XXV, and XLI, of axitinib within the drug product or pharmaceutical composition of the present invention were collected using a Nicolet NXR FT-Raman accessory attached to a Nicolet 6700 FTIR spectrometer equipped with a KBr beamsplitter and a d-TGS KBr detector. The spectrometer is equipped with a 1064 nm Nd:YVO₄ laser and a liquid nitrogen cooled Germanium detector. Tablet samples were analyzed in a static tablet holder, no sample rotation was performed during the experiment. The spectra were collected using 0.5 W of laser power and 100 co-added scans. The collection range was 3700-300 cm⁻¹. The spectra were recorded using 4 cm⁻¹ resolution and Happ-Genzel apodization.

A single spectrum was recorded for each sample, which was intensity normalized prior to peak picking. Peaks were manually identified using the Thermo Nicolet Omnic 7.3a software. Peak position was picked at the peak maximum, and peaks were only identified as such, if there was a slope on each side; shoulders on peaks were not included. API peak intensities will vary with tablet strength and composition. The peak position has been rounded to the nearest whole number using standard practice (0.5 rounds up, 0.4 rounds down). The relative intensity values were grouped into strong (S), medium (M) and weak (W) for the drug products using the following divisions: strong (1-0.75); medium (0.74-0.3) and weak (0.29 and below).

As will be appreciated by the skilled person, the relative intensities of the bands within an active pharmaceutical ingredient Raman peak list may vary due to a number of factors such as the experimental parameters utilized, the type of Raman spectrometer used (FT vs. dispersive), intensity of the excitation source, the particle size and orientation of the material being analyzed, the purity of the material being analyzed, as well as the degree of crystallinity of the sample. The relative intensities of the active pharmaceutical ingredient Raman bands within a drug product sample may vary due to the factors mentioned above as well as additional factors brought about as a result of formulation. Since the majority of a drug product formulation consists of excipients the purity of the crystalline excipient materials within the drug product sample, the degree of crystallinity of the excipients within the drug product sample, the loading of each excipient within the drug product, the identity of the excipients, as well as the active pharmaceutical ingredient loading within the drug product may also cause the relative intensities of Raman bands to vary within a drug product Raman peak list.

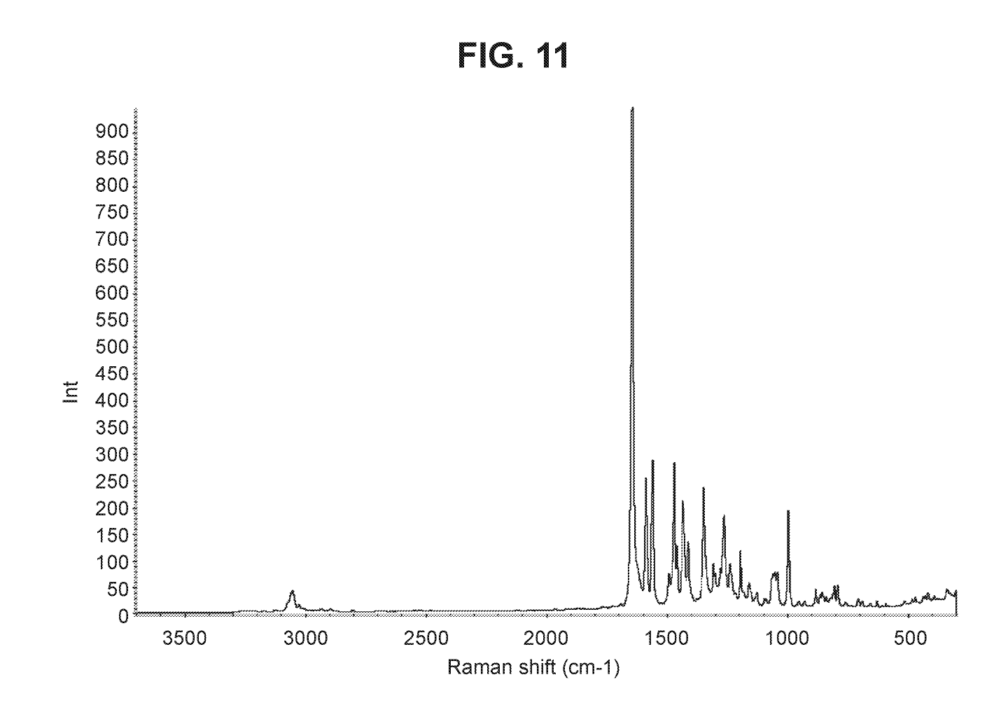

Crystalline Form IV of axitinib API was characterized by the Raman spectrum shown in FIG. 11. The Raman bands of axitinib in drug product, as expressed in wavenumbers, are shown in Table 13.

TABLE 13

| Wavenumber | Relative |
|---|---|
| (cm⁻¹) | Intensity |
| 302 | W |
| 318 | W |
| 329 | W |
| 338 | W |
| 378 | W |
| 391 | W |
| 418 | W |
| 428 | W |
| 437 | W |
| 470 | W |
| 484 | W |
| 515 | W |
| 579 | W |
| 591 | W |
| 607 | W |
| 629 | W |
| 644 | W |
| 656 | W |
| 690 | W |
| 705 | W |
| 762 | W |
| 792 | W |
| 807 | W |
| 813 | W |
| 822 | W |
| 841 | W |
| 851 | W |
| 856 | W |
| 865 | W |
| 884 | W |
| 910 | W |
| 930 | W |
| 945 | W |
| 955 | W |
| 997 | W |
| 1043 | W |
| 1053 | W |
| 1059 | W |
| 1066 | W |
| 1089 | W |
| 1096 | W |
| 1128 | W |
| 1137 | W |
| 1150 | W |
| 1161 | W |
| 1181 | W |
| 1195 | W |
| 1215 | W |
| 1242 | W |
| 1264 | W |
| 1281 | W |
| 1302 | W |
| 1309 | W |
| 1350 | W |
| 1413 | W |
| 1436 | W |
| 1459 | W |
| 1472 | M |
| 1493 | W |
| 1560 | W |
| 1589 | W |
| 1646 | S |
| 2804 | W |
| 2897 | W |
| 2934 | W |
| 3010 | W |
| 3027 | W |
| 3054 | W |
| 3075 | W |
| 3124 | W |

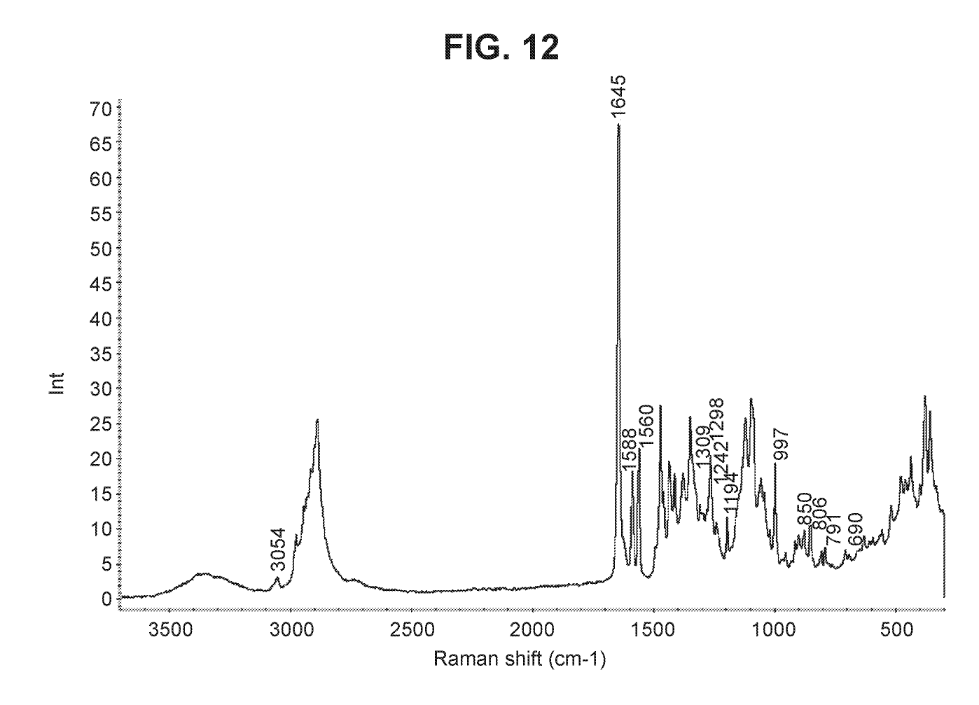

Crystalline Form IV of axitinib in drug product, which was prepared as provided in Example 8, was characterized by the Raman spectrum shown in FIG. 12. The Raman bands of axitinib in drug product, as expressed in wavenumbers, are shown in Table 14.

TABLE 14

| Wavenumber | Relative |
|---|---|
| (cm⁻¹) | Intensity |
| 690 | W |
| 791 | W |
| 806 | W |
| 850 | W |
| 997 | W |
| 1194 | W |
| 1242 | W |
| 1280 | W |
| 1309 | W |
| 1560 | M |
| 1589 | W |
| 1645 | S |
| 3054 | W |

Form IV axitinib in the pharmaceutical composition of the present invention may be identified by a Raman spectrum comprising any one or more of the following Raman shifts expressed as wavenumbers in inverse centimeters: 690±2, 791±2, 806±2, 850±2, 997±2, 1194±2, 1242±2, 1280±2, 1309±2, 1560±2, 1589±2, 1645±2 and 3054±2.

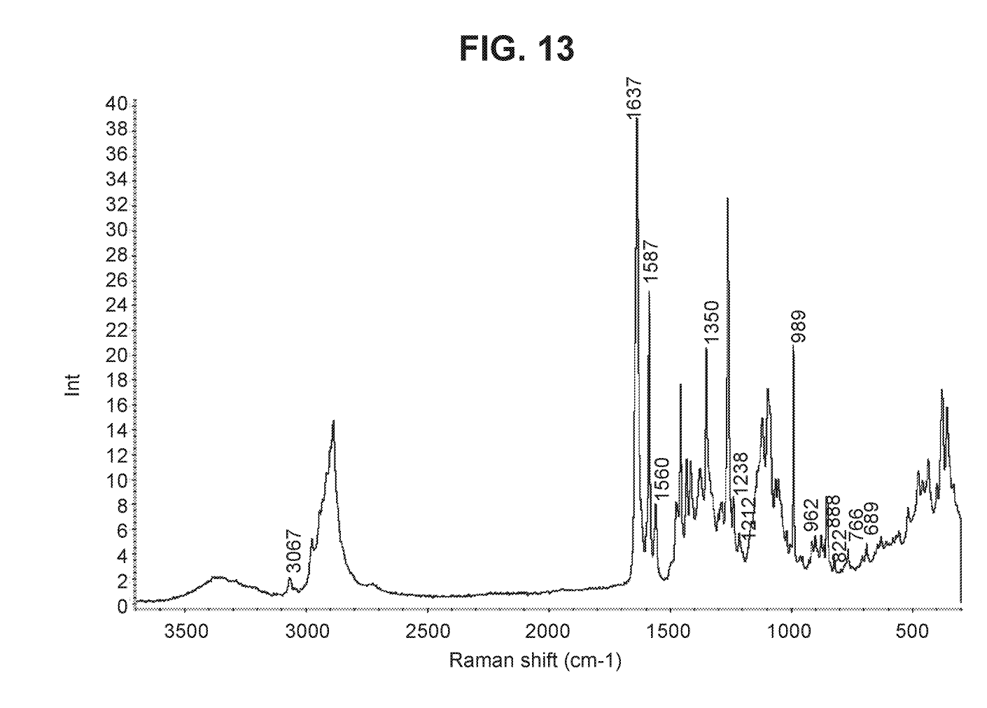

Crystalline Form XXV of axitinib in drug product, which was prepared as provided in Example 8, was characterized by the Raman spectrum shown in FIG. 13. The Raman bands of axitinib in drug product, as expressed in wavenumbers, are shown in Table 15.

TABLE 15

| Wavenumber | Relative |
|---|---|
| (cm⁻¹) | Intensity |
| 689 | W |
| 766 | W |
| 822 | W |
| 866 | W |
| 962 | W |
| 989 | M |
| 1212 | W |
| 1238 | W |
| 1350 | M |
| 1560 | W |
| 1587 | M |
| 1637 | S |
| 3067 | W |

Form XXV axitinib in the pharmaceutical composition of the present invention may be identified by a Raman spectrum comprising any one or more of the following Raman shifts expressed as wavenumbers in inverse centimeters: 689±2, 766±2, 822±2, 866±2, 962±2, 989±2, 1212±2, 1238±2, 1350±2, 1560±2, 1587±2, 1637±2 and 3067±2.

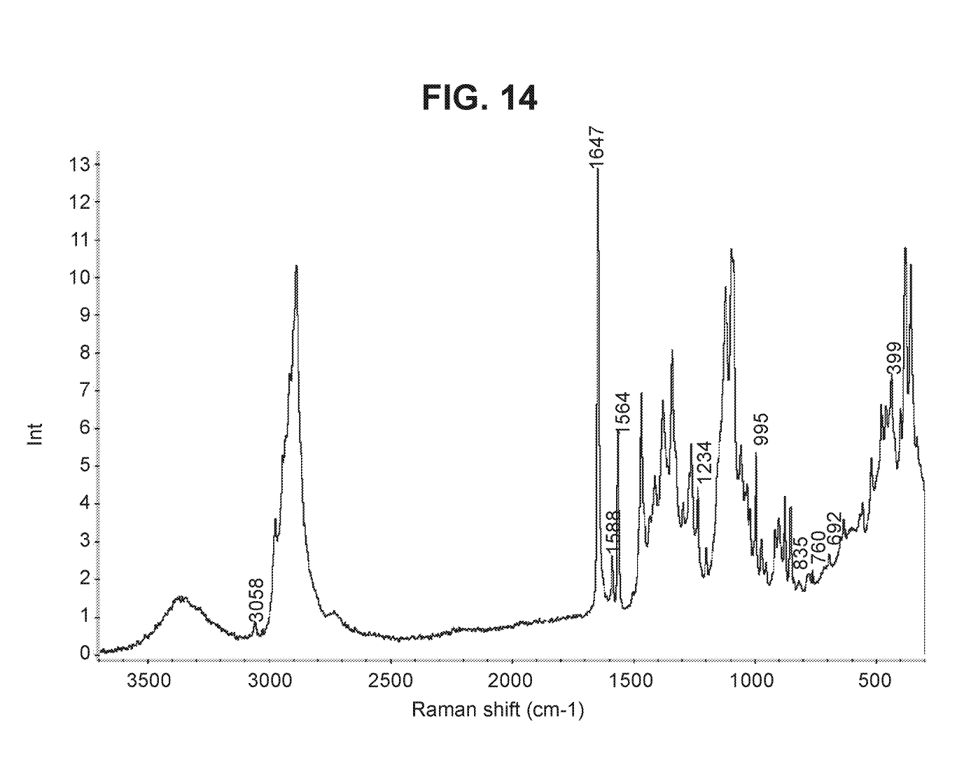

Crystalline Form XLI of axitinib in drug product, which was prepared as provided in Example 8, was characterized by the Raman spectrum shown in FIG. 14. The Raman bands of axitinib in drug product, as expressed in wavenumbers, are shown in Table 16.

TABLE 16

| Wavenumber | Relative |
|---|---|
| (cm⁻¹) | Intensity |
| 399 | M |
| 692 | W |
| 760 | W |
| 835 | W |
| 995 | M |
| 1234 | M |
| 1564 | M |
| 1588 | W |
| 1647 | S |
| 3058 | W |

Form XLI axitinib in the pharmaceutical composition of the present invention may be identified by a Raman spectrum comprising any one or more of the following Raman shifts expressed as wavenumbers in inverse centimeters: 399±2, 692±2, 760±2, 835±2, 995±2, 1234±2, 1564±2, 1588±2, 1647±2 and 3058±2.

### EXAMPLES

The following examples are provided to illustrate the present invention. It should be understood, however, that the invention is not limited to the specific conditions or details described in the examples below.

### Example 1

### Compositions of Opadry® II Blue, Orange, Red, Yellow and Gray Film Coating Systems

The compositions of the Opadry® II Blue and Opadry® II Orange film coating systems are shown in Table 17 below. The compositions of the Opadry® II Red, Opadry® II Yellow and Opadry® II Gray film coating systems are shown in Table 1 above.

TABLE 17
Opadry ® II Blue and Opadry ® II Orange Compositions

| | | Opadry ® II |
|---|---|---|
| | Opadry ® II Blue | Orange |
| Component | (w/w %) | (w/w %) |
| Lactose Monohydrate | 40.000 | 40.000 |
| HPMC 2910/Hypromellose 15 cP | 28.000 | 28.000 |
| Triacetin/Glycerol Triacetate | 8.000 | 8.000 |
| Titanium Dioxide | 21.400 | 21.400 |
| Iron Oxide | N/A | N/A |

### Example 2

### Preparation of Axitinib 1 mg Form IV Blue, Orange, Red, Yellow and Gray Film Coated Tablets

The composition of axitinib 1 mg Form IV blue, orange, red, yellow and gray film coated tablets is shown in Table 18 below.

TABLE 18

| Component | Function | mg/tablet | w/w % |
|---|---|---|---|
| Axitinib Form XLI¹ | API | 1.000 | 1.000 |
| Microcrystalline Cellulose, | Ductile | 63.250 | 63.250 |
| grade 102¹ | Filler | | |
| Lactose Monohydrate | Brittle Filler | 32.000 | 32.000 |
| Croscarmellose Sodium | Disintegrate | 3.000 | 3.000 |
| Magnesium Stearate² | Lubricant | 0.250 | 0.250 |
| Magnesium Stearate³ | Lubricant | 0.500 | 0.500 |
| Core Total (mgW) | | 100.000 | 100.000 |
| Opadry ® II⁴ | Coating | 4.000 | 4.000 |
| Purified Water⁵ | Solvent | N/A | N/A |
| Tablet Total (mgW) | | 104.000 | |

¹The exact amount of axitinib to be weighed will be adjusted for potency. The amount of microcrystalline cellulose will be adjusted accordingly.
²As vegetable grade, added at the blending step
³As vegetable grade, added at the final blending step
⁴The composition is provided in Table 1 and Table 17
⁵Evaporated during processing and does not appear in the final product

The axitinib 1 mg Form IV blue, orange, red, yellow and gray film coated tablets were prepared according to the procedure described below.

### Preparation 1. Preparation of the Axitinib 1 mg Form IV Core Tablets Initial Blend in a Twin Shell Blender

Step 1. Added 3161.5 g microcrystalline cellulose
Step 2. Added 51.0 g axitinib Form IV

### Step 3. Added 1600.0 g Foremost® NF Fast Flo® Lactose (Foremost Farms)

### Step 4. Added 150.0 g Ac-Di-Sol, FMC BioPolymer

Step 5. Blended material in a suitable diffusion mixer

### Mill

Step 1. Milled the blended material through a suitable screening mill

### Blend

Step 1. Blended material in a suitable diffusion mixer

### Final Blend

Step 1. Added 12.5 g magnesium stearate
Step 2. Blended material in a suitable diffusion mixer

### Roll Compaction

Step 1. Utilized a suitable Roller Compactor.

### Mill

Step 1. Milled in a suitable granulator.

### Blend

Step 1. Added 25.0 g magnesium stearate
Step 2. Blended material in a suitable diffusion mixer

### Tableting

Step 1. Compressed into tablets using a suitable tablet press.

### Preparation 2. Film Coating the Axitinib 1 mg Form IV Blue, Orange, Red, Yellow and Gray Film Coated Tablets

The Opadry® II film coating systems were prepared by adding purified water to a vessel. While mixing the contents with a propeller mixer, an Opadry® II film coating system was added and mixed until the solids were well dispersed and free of lumps.”

The core tablets were film coated using the Opadry® II Blue, Opadry® II Orange, Opadry® II Red, Opadry® II Yellow and Opadry® II Gray film coating systems in a Vector LDCS 20/30 coating pan. The target weight gain for the tablets after film coating was 4%.

### Example 3

### Compositions of Opadry® II White and Opadry® Clear Film Coating Systems

The compositions of the Opadry® II White and Opadry® Clear film coating systems are shown in Table 19 below.

TABLE 19

| | Opadry ® II White | Opadry ® Clear |
|---|---|---|
| Component | (w/w %) | (w/w %) |
| Lactose Monohydrate | 40.000 | N/A |
| HPMC 2910/Hypromellose | 28.000 | 90.000 |
| 15 cP | | |
| Triacetin/Glycerol Triacetate | 8.000 | 10.000 |
| Titanium Dioxide | 24.000 | N/A |
| Iron Oxide | N/A | N/A |
| FD&C Yellow #6/ | N/A | N/A |
| Sunset Yellow FCF Aluminum | | |
| Lake | | |
| FD&C Blue#2/ | N/A | N/A |
| Indigo Carmine Aluminum Lake | | |

### Example 4

### Preparation of Axitinib 1 mg Form IV White Film Coated Tablets

The composition of axitinib 1 mg Form IV white film coated tablets is shown in Table 20 below.

TABLE 20

| Component | Function | mg/tablet | w/w % |
|---|---|---|---|
| Axitinib Form XLI¹ | API | 1.000 | 1.000 |
| Microcrystalline Cellulose, | Ductile | 63.250 | 63.250 |
| grade 102¹ | Filler | | |
| Lactose Monohydrate | Brittle Filler | 32.000 | 32.000 |
| Croscarmellose Sodium | Disintegrate | 3.000 | 3.000 |
| Magnesium Stearate² | Lubricant | 0.250 | 0.250 |
| Magnesium Stearate³ | Lubricant | 0.500 | 0.500 |
| Core Total (mgW) | | 100.000 | 100.000 |
| Opadry ® II⁴ | Coating | 4.000 | 4.000 |
| Purified Water⁵ | Solvent | N/A | N/A |
| Tablet Total (mgW) | | 104.000 | |

¹The exact amount of axitinib to be weighed will be adjusted for potency. The amount of microcrystalline cellulose will be adjusted accordingly.
²As vegetable grade, added at the blending step
³As vegetable grade, added at the final blending step
⁴The composition is provided in Table 19 excluding the Opadry ® Clear
⁵Evaporated during processing and does not appear in the final product

The core tablets were manufactured as described in Example 3, Preparation 1. The core tablets were film coated using the Opadry® II White coating system, as described in Table 19 above, in a Vector LDCS 20/30 coating pan. The pan speed was rpm, the solution flow rate was 5 g/minute, the exhaust air temperature was 38-42° C., the pan load was 860 grams of tablets, and the air pressure was 20 PSI. The target weight gain for the tablets after film coating was 4%.

### Example 5

### Photostability Study of 1 mg Axitinib Form IV Drug Product Cores and Blue, White, Orange, Red, Yellow and Gray Film Coated Tablets

A photostability study of axitinib 1 mg Form IV core tablets, blue film coated tablets, orange film coated tablets, red film coated tablets, yellow film coated tablets and gray film coated tablets was performed to determine the degradation propensity of drug substance and drug product. Samples of the tablets tested were prepared as provided in Examples 2 and 4.

The samples were tested under two storage conditions, open dish and closed bottle. For the open dish samples, tablets were spread evenly over the bottom of an uncovered aluminum pan. For the bottle samples, tablets were placed in a 60 cc heat induction sealed high density polyethylene bottle (opaque blue-white with white polypropylene closure; Chevron Phillips Chemical Company).

The samples were also tested in an exposed and control environment. The open dish and closed bottle samples were exposed directly to light and served as the exposed environment. For the control environment, tablets were placed in a capped aluminum pan prior to exposure.

An Atlas Suntest chamber was used to expose the samples to light based on the photostability ICH guidelines as described in “Q1B Photostability Testing of New Drug Substances and Products, Food and Drug Administration—Center for Drug Evaluation and Research, November 1996.” The ICH guidelines state that samples should be exposed to light providing an overall illumination of not less than 1.2 million lux hours and an integrated near ultraviolet energy of not less than 200 watt hours/square meter to allow direct comparisons to be made between the drug substance and drug product.

The samples were analyzed using the HPLC conditions that allowed separation, detection and quantitation of axitinib and photodegradation products.

The percentage of the major photodegradant for the samples is presented in Table 21 below.

No effort was made to protect the tablets from light during manufacture, handling and storage. Therefore, photodegradant formation prior to this experiment was possible. Evidence for photodecomposition is seen in Table 21 where, for example, the red film coating at 2.89% solids had less 2+2 dimer in the HDPE bottle than in the dark control.

For all samples, the results show that there was substantially less photodegradant in the film coated tablets that in the uncoated core tablets. The results also show that the white film coating at 4% solids and the HDPE bottle together were not effective enough to prevent photodecomposition. Tablets with the blue film coating had higher than 0.5% of the 2+2 dimer after direct light exposure, as did the orange film coated tablets at the lower coating level. The amount of photodegradant in the core tablets, and in the orange and blue film coated tablets from HDPE bottles was lower than in the same tablets exposed to light in the open pan. This demonstrated that the HDPE bottles provided some protection from light.

Surprisingly, only the iron oxide film coatings provided superior protection against photodecomposition of axitinib. The iron oxide red, iron oxide yellow and iron oxide gray film coated tablets that were exposed to direct light had amounts of the 2+2 dimer that were similar to the dark control. These coating formulations were shown to be effective without the benefit of the HDPE bottles. Orange and blue film coatings, which do not contain iron oxide, absorbed light as a result of their color but lacked the stabilizing protection of the iron oxide coating formulations for axitinib.

Table 21.

Percentage of the Major Photodegradant in Axitinib 1 mg Form IV Core Tablets, Blue Film Coated Tablets, White Film Coated Tablets, Orange Film Coated Tablets, Red Film Coated Tablets, Yellow Film Coated Tablets and Gray Film Coated

Tablets

| | Coating | 2 + 2 dimer (%) | | |
|---|---|---|---|---|
| | Amounts | Direct light | Sealed HDPE | Dark Control |
| Tablet | (% solids) | (open pan) | bottle | (closed pan) |
| Core | 0.00 | 32.73 | 3.16 | 0.08 |
| Blue FCT | 4.18 | 5.67 | 0.40 | 0.10 |
| | 2.95 | 9.85 | 0.88 | 0.19 |
| White FCT | 4.00 | N/A¹ | 1.45 | 0.09 |
| Orange FCT | 4.78 | 0.32 | 0.06 | 0.06 |
| | 3.25 | 4.53 | 0.10 | 0.11 |
| Red FCT | 5.23 | 0.15 | 0.12 | 0.07 |
| | 2.89 | 0.20 | 0.13 | 0.21 |
| Yellow FCT | 3.56 | 0.15 | 0.16 | 0.08 |
| | 3.40 | 0.09 | 0.09 | 0.10 |
| Gray FCT | 4.19 | 0.12 | 0.08 | 0.12 |
| | 3.39 | 0.13 | 0.09 | 0.08 |

¹The data for the white film coated tablets was generated in an experiment, as described in this Example; however, the only samples tested were tablets in closed HDPE bottles and the dark control.

### Example 6

### Preparation of Axitinib 1 mg Form XLI Core Tablets, White Film Coated Tablets and Red Film Coated Tablets

The compositions of the Opadry® II White and Opadry® Clear film coating systems are shown in Table 19 above. The composition of the Opadry® II Red film coating system is shown in Table 1 above.

The composition of the axitinib 1 mg Form XLI core tablets, white film coated tablets and red film coated tablets is provided in Table 22 below.

TABLE 22
Composition of Axitinib 1 mg Form XLI Tablets

| | | | 1 mg | |
|---|---|---|---|---|
| | | 1 mg | Tablet | 1 mg |
| | | Tablet | (mg/tab) | Tablet |
| Component | | (mg/tab) | White | (mg/tab) |
| Tablet | Function | Core | FCT | Red FCT |
| Axitinib Form XLI¹ | Active | 1.00 | 1.00 | 5.00 |
| Microcrystalline | Ductile Filler | 63.25 | 63.25 | 59.25 |
| Cellulose² | | | | |
| Lactose Monohydrate³ | Brittle Filler | 32.0 | 32.0 | 32.0 |
| Croscarmellose | Disintegrate | 3.0 | 3.0 | 3.0 |
| Sodium⁴ | | | | |
| Magnesium Stearate⁵ | Lubricant | 0.25 | 0.25 | 0.25 |
| Magnesium Stearate⁶ | Lubricant | 0.50 | 0.50 | 0.50 |
| Core Total (mgW) | | 100.0 | 100.0 | 100.0 |
| Opadry ® II White | Coating | | 4.0 | |
| | excipient | | | |
| Opadry ® II Red | Coating | | | 4.0 |
| | excipient | | | |
| Purified Water⁷ | Solvent | | (22.67) | (22.67) |
| Opadry ® II Clear | Coating | | 0.5 | 0.5 |
| | excipient | | | |
| Purified Water⁷ | Solvent | | (9.50) | (9.50) |
| Tablet Total (mgW) | | | 104.5 | 104.5 |

¹Based on 100.0% potency, if potency is different the microcrystalline cellulose will be adjusted.
²Avicel PH102, FMC BioPolymer
³Foremost ® NF Fast Flo ® Lactose, Foremost Farms
⁴Ac-Di-Sol, FMC BioPolymer
⁵Vegetable derived; Malinkrodt; added intragranular
⁶Vegetable derived; Malinkrodt; added extragranular
⁷Volatile

### Preparation 1. Preparation of the Axitinib 1 mg Form XLI Core Tablets Initial Blend in a 10 L Bin Blender

Step 1. Added 1897.5 g microcrystalline cellulose
Step 2. Added 30.0 g axitinib Form XLI

### Step 3. Added 960.0 g Foremost® NF Fast Flo® Lactose (Foremost Farms)

### Step 4. Added 90.0 g Ac-Di-Sol, FMC BioPolymer

Step 5. Blended material in a suitable diffusion mixer

### Mill

Step 1. Milled the blended material through a suitable screening mill

### Blend

Step 1. Blended material in a suitable diffusion mixer

### Final Blend

Step 1. Added 7.50 g magnesium stearate
Step 2. Blended material in a suitable diffusion mixer

### Roll Compaction

Step 1. Utilized a suitable Roller Compactor.

### Blend

Step 1. Added 13.0 g magnesium stearate
Step 2. Blended material in a suitable diffusion mixer

### Tableting

Step 1. Compressed into tablets using a suitable tablet press.
Step 2. Test tablets for hardness, thickness, disintegration and friability

### Preparation 2. Preparation of the Clear Coating for the Axitinib 1 mg Form XLI White and Red Film Coated Tablets

Step 1. Mix Solution: Added 501.67 g deionized water

### Step 2. Mix Solution: Added 26.40 g Opadry® II Clear

Step 3. Mixed until a solution was formed.

### Preparation 3. Preparation of the White Film Coating for the Axitinib 1 mg Form XLI White Film Coated Tablets

The Opadry® II White film coating system was prepared by adding purified water to a vessel. While mixing the contents with a propeller mixer, Opadry® II White was added and mixed until the solids were well dispersed and free of lumps.”

### Preparation 4. Preparation of the Red Film Coating for the Axitinib 1 mg Form XLI Red Film Coated Tablets

Step 1. Mix Suspension: Added 598.53 g deionized water

### Step 2. Mix Suspension: Added 105.61 g Opadry® II Red

Step 3. Mixed for >45 minutes.

### Preparation 5. Preparation of the Axitinib 1 mg Form XLI White Film Coated Tablets

The core tablets were film coated using the Opadry® II White film coating system in a suitable coating pan. The target weight gain for the tablets after film coating was 4%.

### Preparation 6. Preparation of the Axitinib 1 mg XLI Red Film Coated Tablets

The core tablets were film coated using the Opadry® II Red film coating systems in a Vector LDCS 20/30 coating pan. The target weight gain for the tablets after film coating was 4%.

### Example 7

### Photostability Study of 1 mg Axitinib Form XLI Drug Product Cores and Film Coated Tablets

A photostability study of axitinib 1 mg Form XLI core tablets, white film coated tablets and red film coated tablets was performed to determine the degradation propensity of drug substance and drug product. Samples of axitinib 1 mg Form XLI core tablets, white film coated tablets and red film coated tablets were prepared as provided in Example 6.

The samples were tested under two storage conditions, open dish and closed bottle. For the open dish samples, tablets were spread evenly over the bottom of a shallow glass dish. For the bottle samples, tablets were placed in a square high density polyethylene bottle with a squeeze and turn closure. The closure was not heat-sealed.

The samples were also tested in an exposed and control environment. The open dish and closed bottle samples were exposed directly to light and served as the exposed environment. For the control environment, tablets were wrapped in aluminum foil prior to exposure.

The samples were exposed to light based on the photostability ICH guidelines as described in “Q1B Photostability Testing of New Drug Substances and Products, Food and Drug Administration—Center for Drug Evaluation and Research, November 1996.” An Atlas Suntest XLS+ instrument was used to expose the samples to UV and fluorescent light. The photostability study was designed to expose the samples to an exposure equivalent to 1×ICH and 5×ICH for fluorescents. Due to the nature of the light box, final exposure was equivalent to 1×ICH and 5×ICH for fluorescent and 2.5×ICH and 12.5×ICH for UV.

See Table 23 for the sample configurations and light conditions tested.

The samples were analyzed using the HPLC conditions that allowed separation, detection and quantitation of axitinib and photodegradation products.

The percentage of the major photodegradants for the samples is presented in Table 23 below.

For all samples, the results show that there was substantially less photodegradant in the film coated tablets that in the uncoated core tablets. The amount of photodegradant in the core tablets and in the white film coated tablets from HDPE bottles was lower than in the same tablets exposed to light in the open pan. This demonstrated that the HDPE bottles provided some protection from light.

Surprisingly, only the iron oxide red film coating provided superior protection against photodecomposition of axitinib. The iron oxide red film coated tablets that were exposed to direct light had amounts of the 2+2 dimer that were similar to the dark control. This coating formulation was shown to be effective without the benefit of the HDPE bottles.

It is noted that results obtained for the 1 mg tablets should not be significantly different to those obtained for the 5 mg tablets due to the nature of the light exposure and its lack of dependency on drug to excipient ratios.

TABLE 23
Percentage of Major Photodegradants in Axitinib 1 mg Form XLI Core Tablets,
White Film Coated Tablets and Red Film Coated Tablets

| | | UV/Fluorescent | % cis-isomer | | % asymmetric dimer | |
|---|---|---|---|---|---|---|
| | | Exposure | Average (n = 1) | | Average (n = 1) | |
| Tablet | Storage | Equivalence | Control | Exposed | Control | Exposed |
| Core | Open | 2.5xICH/1xICH | 0.20 | 1.09 | 0.23 | 4.10 |
| | Bottle | 2.5xICH/1xICH | ≦0.05 | 0.09 | ≦0.05 | 0.08 |
| White | Open | 2.5xICH/1xICH | ≦0.05 | 0.67 | ≦0.05 | 0.87 |
| FCT | Bottle | 2.5xICH/1xICH | ≦0.05 | ≦0.05 | ≦0.05 | ≦0.05 |
| Red | Open | 2.5xICH/1xICH | ≦0.05 | ≦0.05 | ≦0.05 | ≦0.05 |
| FCT | Bottle | 2.5xICH/1xICH | ≦0.05 | ≦0.05 | ≦0.05 | ≦0.05 |
| Core | Open | 12.5xICH/5xICH | 0.20 | 1.22 | 0.23 | 4.59 |
| | Bottle | 12.5xICH/5xICH | ≦0.05 | 0.14 | ≦0.05 | 0.26 |
| White | Open | 12.5xICH/5xICH | ≦0.05 | 0.98 | ≦0.05 | 2.53 |
| FCT | Bottle | 12.5xICH/5xICH | ≦0.05 | 0.07 | ≦0.05 | 0.10 |
| Red | Open | 12.5xICH/5xICH | ≦0.05 | ≦0.05 | ≦0.05 | ≦0.05 |
| FCT | Bottle | 12.5xICH/5xICH | ≦0.05 | ≦0.05 | ≦0.05 | ≦0.05 |

### Example 8

### Preparation of Axitinib 5 mg Form IV, Form XXV and Form XLI Red Film Coated Tablets for Solid-State Evaluation

The compositions of axitinib 5 mg Form IV, Form XXV and Form XLI red film coated tablets used for solid-state evaluation are shown in Table 24 below.

Axitinib 5 mg film coated tablets were prepared using crystalline Forms IV, XXV, and XLI. The API loading was adjusted to prepare a 5 mg active level in the tablet formulation based on the API potency. The microcrystalline cellulose loading was adjusted to compensate for changes in API level in order to maintain a common tablet weight of approximately 183 mg.

TABLE 24
Compositions of Axitinib 5 mg Film Coated Tablets

| | Form IV | Form XXV | Form XLI |
|---|---|---|---|
| Component | (mg/tab) | (mg/tab) | (mg/tab) |
| Axitinib | 5.10¹ | 5.00² | 5.00² |
| Microcrystalline Cellulose³ | 108.86 | 109.86 | 108.96 |
| Lactose Monohydrate⁴ | 54.48 | 54.48 | 54.48 |
| Croscarmellose Sodium⁵ | 5.25 | 5.25 | 5.25 |
| Magnesium Stearate | 1.32 | 0.44 | 0.44 |
| (Intragranular) | | | |
| Magnesium Stearate | N/A | 0.88 | 0.88 |
| (Intergranular) | | | |
| Core Total | 175.00 | 175.00 | 175.00 |
| Opadry II ® Red | 7.00 | 7.00 | 7.00 |
| Opadry I Clear⁶ | 0.88 | 0.88 | 0.88 |
| Tablet Total (mg) | 182.88 | 182.88 | 182.88 |

¹Based on 98.4% potency.
²Based on 100.0% potency.
³Avicel PH102, FMC BioPolymer
⁴Foremost ® NF Fast Flo ® Lactose, Foremost Farms
⁵Ac-Di-Sol, FMC BioPolymer
⁶Colorcon Opadry I Clear (lot YS-2-19114-A)

### Preparation 1. Preparation of the Axitinib 5 mg Form IV Film Coated Tablets

Axitinib 5 mg Form IV film coated tablets were prepared by adding 2488 grams microcrystalline cellulose, 116 grams axitinib Form IV, 1245 grams Foremost® NF Fast Flo® Lactose, and 120 grams Ac-Di-Sol to a suitable blender and blending for a suitable period of time. The blend was milled through a suitable screening mill and then blended in a suitable diffusion mixer. 10.0 grams of intragranular magnesium stearate was added to the milled blend and the mixture blended in a suitable diffusion mixer. The blend was roller compacted and then milled in a suitable granulator. The milled material was then added to a suitable blender with an amount of extragranular magnesium stearate and blended for a suitable period of time. The blend was then tabletted using a suitable tablet press. The resulting tablets were first film coated with Red Opadry® II. The target Weight gain was 4%. The resulting film coated tablets were then film coated with Opadry® I. The target Weight gain was 0.5%.

### Preparation 2. Preparation of the Axitinib 5 mg Form XXV Film Coated Tablets

Axitinib 5 mg Form XXV film coated tablets were prepared according to the procedure described in Preparation 1 above, with the exception that axitinib Form XXV was used in place of axitinib Form IV.

### Preparation 3. Preparation of the Axitinib 5 mg Form XLI Film Coated Tablets

Axitinib 5 mg Form XLI film coated tablets were prepared by adding 1868 grams microcrystalline cellulose, 86 grams axitinib Form XLI, 934 grams Foremost® NF Fast Flo® Lactose, and 90 grams Ac-Di-Sol to a suitable blender and blending for a suitable period of time. The blend was milled through a suitable screening mill and then blended in a suitable diffusion mixer. 7.5 grams of intragranular magnesium stearate was added to the milled blend and the mixture blended in a suitable diffusion mixer. The blend was roller compacted and then milled in a suitable granulator. The milled material was then added to a suitable blender with an amount of extragranular magnesium stearate and blended for a suitable period of time. The blend was then tabletted using a suitable tablet press. The resulting tablets were first film coated with Red Opadry® II. The target Weight gain was 4%. The resulting film coated tablets were then film coated with Opadry® I. The target Weight gain was 0.5%.

1. A pharmaceutical composition comprising a core and a coating, the core comprising N-methyl-2-[3-((E)-2-pyridin-2-yl-vinyl)-1H-indazol-6-ylsulfanyl]-benzamide or a pharmaceutically acceptable salt thereof and excipients, and the coating comprising a metal oxide.

2. The pharmaceutical composition of claim 1, wherein the coating further comprises a filler, a polymer, a plasticizer, or an opacifier, or combinations thereof.

3. The pharmaceutical composition of claim 2, wherein the coating further comprises a colorant.

4. The pharmaceutical composition of claim 1, wherein the metal oxide comprises iron oxide.

5. The pharmaceutical composition of claim 1, wherein the coating is selected from the group consisting of Opadry II Red®, Opadry II Yellow®, and Opadry II Gray®.

6. The pharmaceutical composition of claim 1, wherein the coating is Opadry II Red®.

7. The pharmaceutical composition of claim 1, wherein the composition is a film coated tablet.

8. A pharmaceutical composition comprising N-methyl-2-[3-((E)-2-pyridin-2-yl-vinyl)-1H-indazol-6-ylsulfanyl]-benzamide or a pharmaceutically acceptable salt thereof and excipients, wherein the pharmaceutical composition comprises at least one compound selected from the group consisting of

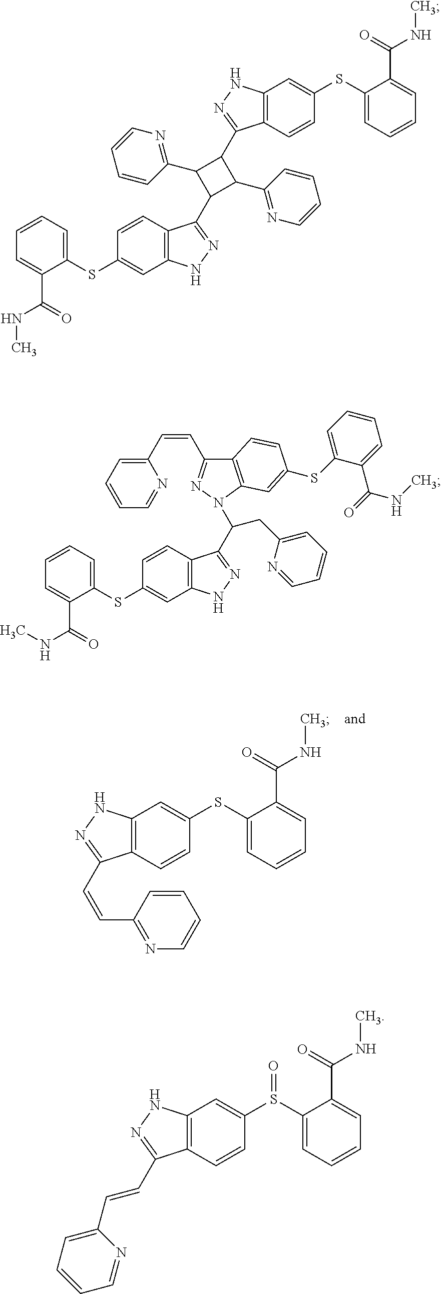 [SMILES: CNC(=O)c1ccccc1Sc1ccc2c(C3C(c4ccccn4)C(c4n[nH]c5cc(Sc6ccccc6C(=O)NC)ccc45)C3c3ccccn3)n[nH]c2c1 ; CNC(=O)c1ccccc1Sc1ccc2c(C(Cc3ccccn3)n3nc(/C=C\c4ccccn4)c4ccc(Sc5ccccc5C(=O)NC)cc43)n[nH]c2c1 ; CNC(=O)c1ccccc1Sc1ccc2c(/C=C\c3ccccn3)n[nH]c2c1 ; "and" ; CNC(=O)c1ccccc1S(=O)c1ccc2c(/C=C/c3ccccn3)n[nH]c2c1]

9. A pharmaceutical composition comprising N-methyl-2-[3-((E)-2-pyridin-2-yl-vinyl)-1H-indazol-6-ylsulfanyl]-benzamide or a pharmaceutically acceptable salt thereof and excipients, wherein the pharmaceutical composition comprises less than about 1.0 weight percent of a compound, which is

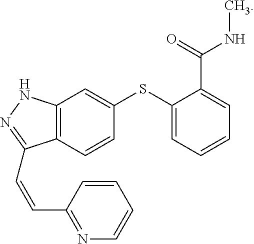 [SMILES: CNC(=O)c1ccccc1Sc1ccc2c(/C=C\c3ccccn3)n[nH]c2c1]

10. A compound, which is 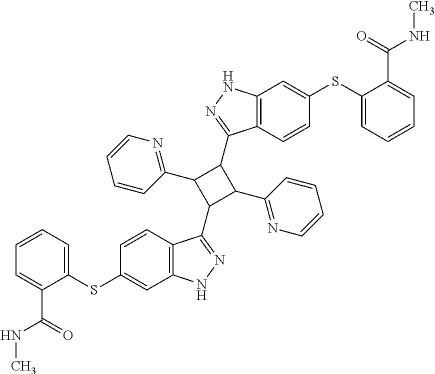 [SMILES: CNC(=O)c1ccccc1Sc1ccc2c(C3C(c4ccccn4)C(c4n[nH]c5cc(Sc6ccccc6C(=O)NC)ccc45)C3c3ccccn3)n[nH]c2c1]
or a pharmaceutically acceptable salt thereof.

11. A compound, which is 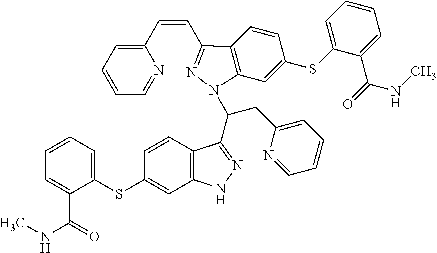
or a pharmaceutically acceptable salt thereof.

12. The pharmaceutical composition of claim 1, wherein the pharmaceutical composition comprises about 1 mg of N-methyl-2-[3-((E)-2-pyridin-2-yl-vinyl)-1H-indazol-6-ylsulfanyl]-benzamide and:
a. about 89 weight percent to about 97 weight percent of at least one filler;
b. about 2 weight percent to about 5 weight percent of a disintegrant;
c. about 0.25 weight percent to about 5 weight percent of a lubricant; and
d. about 1 weight percent to about 8 weight percent of the coating, based on the total weight of the pharmaceutical composition.

13. The pharmaceutical composition of claim 1, wherein the pharmaceutical composition comprises about 3 mg, about 5 mg or about 7 mg of N-methyl-2-[3-((E)-2-pyridin-2-yl-vinyl)-1H-indazol-6-ylsulfanyl]-benzamide and:
a. about 87 weight percent to about 95 weight percent of at least one filler;
b. about 2 weight percent to about 5 weight percent of a disintegrant;
c. about 0.25 weight percent to about 5 weight percent of a lubricant; and
d. about 1 weight percent to about 9 weight percent of the coating, based on the total weight of the pharmaceutical composition.

14. The pharmaceutical composition of claim 1, wherein the pharmaceutical composition comprises about 1 mg of N-methyl-2-[3-((E)-2-pyridin-2-yl-vinyl)-1H-indazol-6-ylsulfanyl]-benzamide and:
a. about 20 weight percent to about 90 weight percent microcrystalline cellulose;
b. about 10 weight percent to about 85 weight percent lactose monohydrate;
c. about 2 weight percent to about 5 weight percent croscarmellose sodium;
d. about 0.25 weight percent to about 5 weight percent magnesium stearate; and
e. about 1 weight percent to about 8 weight percent of the coating, based on the total weight of the pharmaceutical composition.

15. The pharmaceutical composition of claim 1, wherein the pharmaceutical composition comprises about 3 mg, about 5 mg or about 7 mg of N-methyl-2-[3-((E)-2-pyridin-2-yl-vinyl)-1H-indazol-6-ylsulfanyl]-benzamide and:
a. about 20 weight percent to about 90 weight percent microcrystalline cellulose;
b. about 10 weight percent to about 85 weight percent lactose monohydrate;
c. about 2 weight percent to about 5 weight percent croscarmellose sodium;
d. about 0.25 weight percent to about 5 weight percent magnesium stearate; and
e. about 1 weight percent to about 8 weight percent of the coating, based on the total weight of the pharmaceutical composition.

16. The pharmaceutical composition of claim 1, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 8.8±0.1, 12.0±0.1, 14.5±0.1, 15.7±0.1 and 19.1±0.1.

17. The pharmaceutical composition of claim 1, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a solid state nuclear magnetic resonance comprising the following ¹³C chemical shifts expressed in parts per million: 154.2±0.2, 143.3±0.2, 121.3±0.2 and 27.8±0.2.

18. The pharmaceutical composition of claim 1, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a Raman spectrum comprising any one of the following Raman shifts expressed as wavenumbers in inverse centimeters: 791±2, 806±2, 850±2, 1194±2, 1242±2, 1280±2, 1309±2 and 3054±2.

19. The pharmaceutical composition of claim 1, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 8.8±0.1 and 15.7±0.1 and a solid state nuclear magnetic resonance comprising the following ¹³C chemical shifts expressed in parts per million: 154.2±0.2, 143.3±0.2, 121.3±0.2 and 27.8±0.2.

20. The pharmaceutical composition of claim 1, wherein the N-methyl-2-[3-((E)-2-pyridin-2-yl-vinyl)-1H-indazol-6-ylsulfanyl]-benzamide is Form IV N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 8.8±0.1 and 15.7±0.1 and a Raman spectrum comprising any one of the following Raman shifts expressed as wavenumbers in inverse centimeters: 791±2, 806±2, 850±2, 1194±2, 1242±2, 1280±2, 1309±2 and 3054±2.

21. The pharmaceutical composition of claim 1, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 11.5±0.1, 11.9±0.1, 14.8±0.1 and 15.6±0.1.

22. The pharmaceutical composition of claim 1, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a solid state nuclear magnetic resonance comprising the following ¹³C chemical shifts expressed in parts per million: 142.6±0.2, 133.7±0.2, 121.4±0.2 and 119.8±0.2.

23. The pharmaceutical composition of claim 1, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a Raman spectrum comprising any one of the following Raman shifts expressed as wavenumbers in inverse centimeters: 835±2, 1234±2, 1564±2 and 3058±2.

24. The pharmaceutical composition of claim 1, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 11.5±0.1 and 11.9±0.1 and a solid state nuclear magnetic resonance comprising the following ¹³C chemical shifts expressed in parts per million: 142.6±0.2, 133.7±0.2, 121.4±0.2 and 119.8±0.2.

25. The pharmaceutical composition of claim 1, wherein the N-methyl-2-[3-((E)-2-pyridin-2-yl-vinyl)-1H-indazol-6-ylsulfanyl]-benzamide is Form XLI N-methyl-2-[3-((E)-2-pyridin-2-yl-vinyl)-1H-indazol-6-ylsulfanyl]-benzamide having a powder X-ray diffraction pattern comprising the following 2θ values measured using CuKₐ radiation (λ=1.54056 Å): 11.5±0.1 and 11.9±0.1 and a Raman spectrum comprising any one of the following Raman shifts expressed as wavenumbers in inverse centimeters: 835±2, 1234±2, 1564±2 and 3058±2.

26. The pharmaceutical composition of claim 1, wherein photodegradation of the N-methyl-2-[3-((E)-2-pyridin-2-yl-vinyl)-1H-indazol-6-ylsulfanyl]-benzamide or a pharmaceutically acceptable salt thereof, is less than about 1% as measured by the International Conference on Harmonisation of Technical Requirements for Registration of Pharmaceuticals for Human Use guideline, Q1B Photostability Testing of New Drug Substances and Products, published on November 1996.

27. The pharmaceutical composition of claim 1, wherein photodegradation of the N-methyl-2-[3-((E)-2-pyridin-2-yl-vinyl)-1H-indazol-6-ylsulfanyl]-benzamide or a pharmaceutically acceptable salt thereof, is less than about 0.05% as measured by the International Conference on Harmonisation of Technical Requirements for Registration of Pharmaceuticals for Human Use guideline, Q1B Photostability Testing of New Drug Substances and Products, published on November 1996.

28. The pharmaceutical composition of claim 1, wherein photodegradation of the N-methyl-2-[3-((E)-2-pyridin-2-yl-vinyl)-1H-indazol-6-ylsulfanyl]-benzamide or a pharmaceutically acceptable salt thereof, is less than about 0.01% as measured by the International Conference on Harmonisation of Technical Requirements for Registration of Pharmaceuticals for Human Use guideline, Q1B Photostability Testing of New Drug Substances and Products, published on November 1996.
